(19) United States
(12) Patent Application Publication
(10) Pub. No.: US 2009/0149506 A1
(43) Pub. Date: Jun. 11, 2009

(54) INSECTICIDAL COMPOSITIONS WITH IMPROVED EFFECT

(75) Inventors: Christian Funke, Leichlingen (DE); Reiner Fischer, Monheim (DE); Peter Marczok, Koln (DE); Rolf Pontzen, Leichlingen (DE); Udo Reckmann, Koln (DE); Christian Arnold, Langenfeld (DE); Erich Sanwald, Kiel (DE)

Correspondence Address: Baker Donelson Bearman, Caldwell & Berkowitz, PC, 555 Eleventh Street, NW, Sixth Floor, Washington, DC, 20004, US

(73) Assignee: Bayer CropScience AG, Monheim Am Rhein (DE)

(21) Appl. No.: 12/097,044

(22) PCT Filed: Nov. 30, 2006
(86) PCT No.: PCT/EP2006/011471

§ 371 Date: Sep. 19, 2008

(30) Foreign Application Priority Data
Dec. 13, 2005 (DE) 102005059470.0

(51) Int. Cl. A61K 31/4439

(52) U.S. Cl. 514/341

(57) ABSTRACT

The present invention relates to increasing the activity of crop protection compositions comprising anthranilic acid diamides through the addition of ammonium salts and/or phosphonium salts or through the addition of ammonium or phosphonium salts and penetrants, to the corresponding compositions, to processes for preparing them and to their use in crop protection.

The present invention relates to increasing the activity of crop protection compositions comprising anthranilic acid diamides (anthranilamides) through the addition of ammonium salts and/or phosphonium salts or through the addition of ammonium or phosphonium salts and penetrants, to the corresponding compositions, to processes for preparing them and to their use in crop protection.

All inhibitors according to the invention of the nicotinic acetylcholine receptor are already known as agents for controlling animal pests, in particular insects, and can be prepared by processes described in the prior art. The activity of these compounds is good; however, in particular at low application rates and concentrations, it is not always entirely satisfactory. Furthermore, the compatibility of these compounds with plants is not always sufficient. There is therefore a need for increasing the activity of the crop protection compositions comprising the compounds.

The anthranilic acid diamides of the formula (I) are likewise known compounds which are known from the following publications, or comprised by them:

NL-A 9202078, WO 01/70671, WO 02/094791, JP-A 2003-212834, WO 03/015519, WO 03/016284, WO 03/015518, WO 03/015519, WO 03/024222, WO 03/016282, WO 03/016283, WO 03/062226, WO 03/027099, WO 04/027042, WO 04/033468.

Mixtures of anthranilic acid diamides with other active compounds are known from WO 05/107468. This publication also described mixtures with petroleum oils.

The anthranilic acid diamides can by summarized by the formula (I):

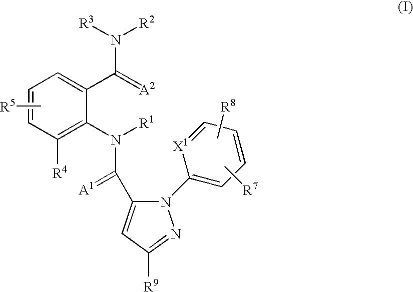

in which
A¹ and A² independently of one another represent oxygen or sulphur,
X¹ represents N or CR¹⁰,
R¹ represents hydrogen or represents in each case optionally mono- or polysubstituted C₁-C₆-alkyl, C₂-C₆-alkenyl, C₂-C₆-alkynyl or C₃-C₆-cycloalkyl, where the substituents independently of one another may be selected from the group consisting of R⁶, halogen, cyano, nitro, hydroxyl, C₁-C₄-alkoxy, C₁-C₄-alkylthio, C₁-C₄-alkylsulphinyl, C₁-C₄-alkylsulphonyl, C₂-C₄-alkoxycarbonyl, C₁-C₄-alkylamino, C₂-C₈-dialkylamino, C₃-C₆-cycloalkylamino, (C₁-C₄-alkyl)C₃-C₆-cycloalkylamino and R¹¹,
R² represents hydrogen, C₁-C₆-alkyl, C₂-C₆-alkenyl, C₂-C₆-alkynyl, C₃-C₆-cycloalkyl, C₁-C₄-alkoxy, C₁-C₄-alkylamino, C₂-C₈-dialkylamino, C₃-C₆-cycloalkylamino, C₂-C₆-alkoxycarbonyl or C₂-C₆-alkylcarbonyl,
R³ represents hydrogen, R¹¹ or represents in each case optionally mono- or polysubstituted C₁-C₆-alkyl, C₂-C₆-alkenyl, C₂-C₆-alkynyl, C₃-C₆-cycloalkyl, where the substituents independently of one another may be selected from the group consisting of R⁶, halogen, cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₂-C₆-alkoxycarbonyl, C₂-C₆-alkylcarbonyl, C₃-C₆-trialkylsilyl, R¹¹, phenyl, phenoxy or a 5- or 6-membered heteroaromatic ring, where each phenyl, phenoxy and 5- or 6-membered heteroaromatic ring may optionally be substituted and where the substituents independently of one another may be selected from the group consisting of one to three radicals W and one or more radicals R¹², or
R² and R³ may be attached to one another and form the ring M,
R⁴ represents hydrogen, C₁-C₆-alkyl, C₂-C₆-alkenyl, C₂-C₆-alkynyl, C₃-C₆-cycloalkyl, C₁-C₆-haloalkyl, C₂-C₆-haloalkenyl, C₂-C₆-haloalkynyl, C₃-C₆-halocycloalkyl, halogen, cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, C₃-C₆-cycloalkylamino, C₃-C₆-trialkylsilyl or represents in each case optionally mono- or polysubstituted phenyl, benzyl or phenoxy, where the substituents independently of one another may be selected from the group consisting of C₁-C₄-alkyl, C₂-C₄-alkenyl, C₂-C₄-alkynyl, C₃-C₆-cyclalkyl, C₁-C₄-haloalkyl, C₂-C₄-haloalkenyl, C₂-C₄-haloalkynyl, C₃-C₆-halocycloalkyl, halogen, cyano, nitro, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylamino, C₂-C₈-dialkylamino, C₃-C₆-cycloalkylamino, C₃-C₆-(alkyl)cycloalkylamino, C₂-C₄-alkylcarbonyl, C₂-C₆-alkoxycarbonyl, C₂-C₆-alkylaminocarbonyl, C₃-C₈-dialkylaminocarbonyl and C₃-C₆-trialkylsilyl,
R⁵ and R⁸ each independently of one another represent hydrogen, cyano, halogen or represent in each case optionally substituted C₁-C₄-alkyl, C₁-C₄-haloalkyl, R¹², G, J, —OJ, —OG, —S(O)ₚ-J, —S(O)ₚ-G, —S(O)ₚ-phenyl, where the substituents independently of one another may be selected from the group consisting of one to three radicals W or of R², C₁-C₁₀-alkyl, C₂-C₆-alkenyl, C₂-C₆-alkynyl, C₁-C₄-alkoxy or C₁-C₄-alkylhio, where each substituent may be substituted by one or more substituents independently of one another selected from the group consisting of G, J, R⁶, halogen, cyano, nitro, amino, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, C₃-C₆-trialkylsilyl, phenyl and phenoxy, where each phenyl or phenoxy ring may optionally be substituted and where the substituents independently of one another may be selected from the group consisting of one to three radicals W and one or more radicals R¹²,
G in each case independently represents a 5- or 6-membered non-aromatic carbocyclic or heterocyclic ring which may optionally contain one or two ring members from the group consisting of C(═O), SO and S(═O)₂ and which may optionally be substituted by one to four substituents independently of one another selected from the group consisting of C₁-C₂-alkyl, halogen, cyano, nitro and C₁-C₂-alkoxy, or independently represents C₂-C₆-alkenyl, C₂-C₆-alkynyl, C₃-C₇-cycloalkyl, (cyano)C₃-C₇-cycloalkyl, (C₁-C₄-alkyl)C₃-C₆-cycloalkyl, (C₃-C₆-cycloalkyl)C₁-C₄-alkyl, where each cycloalkyl, (alkyl)cycloalkyl and (cycloalkyl)alkyl may optionally be substituted by one or more halogen atoms,
J in each case independently represents an optionally substituted 5- or 6-membered heteroaromatic ring, where the substituents independently of one another may be selected from the group consisting of one to three radicals W and one or more radicals R¹²,
R⁶ independently represents —C(=E¹)R¹⁹, -LC(=E¹)R¹⁹, —C(=E¹)LR¹⁹, -LC(=E¹)LR¹⁹, —OP(═O)(OR¹⁹)₂, —SO₂LR¹⁸ or -LSO₂LR¹⁹, where each E¹ independently represents O, S, N—R¹⁵, N—OR¹⁵, N—N(R¹⁵)₂, N—S═O, N—CN or N—NO₂,
R⁷ represents hydrogen, C₁-C₄-alkyl, C₁-C₄-haloalkyl, halogen, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkyl-sulphinyl, C₁-C₄-haloalkylsulphonyl,
R⁹ represents C₁-C₄-haloalkyl, C₁-C₄-haloalkoxy, C₁-C₄-haloalkylsulphinyl or halogen,
R¹⁰ represents hydrogen, C₁-C₄-alkyl, C₁-C₄-haloalkyl, halogen, cyano or C₁-C₄-haloalkoxy,
R¹¹ in each case independently represents in each case optionally mono- to trisubstituted C₁-C₆-alkylthio, C₁-C₆-alkylsulphenyl, C₁-C₆-haloalkylhio, C₁-C₆-haloalkylsulphenyl, phenylthio or phenylsulphenyl, where the substituents independently of one another may be selected from the list W, —S(O)ₙN(R⁶)₂, —C(═O)R¹³, -L(C═O)R¹⁴, —S(C═O)LR¹⁴, —C(═O)LR¹³, —S(O)ₙNR¹³C(═O)R¹³, —S(O)ₙNR¹³C(═O)LR¹⁴ and —S(O)ₙNR¹³S(O)₂LR⁹⁴,
L in each case independently represents O, NR¹⁸ or S,
R¹² in each case independently represents —B(OR¹⁷)₂, amino, SH, thiocyanato, C₃-C₈-trialkylsilyloxy, C₁-C₄-alkyl disulphide, —SF₅, —C(=E¹)R¹⁹, -LC(=E¹)R¹⁹, —C(=E¹)LR¹⁹, -LC(=E¹)LR¹⁹, —OP(═O)(OR¹⁹)₂, —SO₂LR¹⁹ or -LSO₂LR¹⁹,
Q represents O or S,
R¹³ in each case independently represents hydrogen or represents in each case optionally mono- or polysubstituted C₁-C₆-alkyl, C₂-C₆-alkenyl, C₂-C₆-alkynyl or C₃-C₆-cycloalkyl, where the substituents independently of one another may be selected from the group consisting of R⁶, halogen, cyano, nitro, hydroxyl, C₁-C₄-alkoxy, C₁-C₄-alkylsulphinyl, C₁-C₄-alkylsulphonyl, C₁-C₄-alkylamino, C₂-C₈-dialkylamino, C₃-C₆-cycloalkylamino and (C₁-C₄-alkyl)C₃-C₆-cycloalkylamino,
R¹⁴ in each case independently represents in each case optionally mono- or polysubstituted C₁-C₂₀-alkyl, C₂-C₂₀-alkenyl, C₂-C₂₀-alkynyl or C₃-C₆-cycloalkyl, where the substituents independently of one another may be selected from the group consisting of R⁶, halogen, cyano, nitro, hydroxyl, C₁-C₄-alkoxy, C₁-C₄-alkylsulphinyl, C₁-C₄-alkylsulphonyl, C₁-C₄-alkylamino, C₂-C₈-dialkylamino, C₃-C₆-cycloalkylamino and (C₁-C₄-alkyl)C₃-C₆-cycloalkylamino or represents optionally substituted phenyl, where the substituents independently of one another may be selected from the group consisting of one to three radicals W and one or more radicals R¹²,
R¹⁵ in each case independently represents hydrogen or represents in each case optionally mono- or polysubstituted C₁-C₆-haloalkyl or C₁-C₆-alkyl, where the substituents independently of one another may be selected from the group consisting of cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, C₂-C₆-alkoxycarbonyl, C₂-C₆-alkylcarbonyl, C₃-C₆-trialkylsilyl and optionally substituted phenyl, where the substituents independently of one another may be selected from the group consisting of one to three radicals W and one or more radicals R¹², or N(R⁵)₂ represents a cycle which forms the ring M,
R¹⁶ represents C₁-C₁₂-alkyl or C₁-C₁₂-haloalkyl, or N(R¹⁶)₂ represents a cycle which forms the ring M,
R¹⁷ in each case independently represents hydrogen or C₁-C₄-alkyl, or B(OR¹⁷)₂ represents a ring in which the two oxygen atoms are linked via a chain of two or three carbon atoms which are optionally substituted by one or two substituents independently of one another selected from the group consisting of methyl and C₂-C₆-alkoxycarbonyl,
R¹⁸ in each case independently represents hydrogen, C₁-C₆-alkyl or C₁-C₆-haloalkyl, or N(R¹³)(R¹⁸) represents a cycle which forms the ring M,
R¹⁹ in each case independently of one another represents hydrogen or represents in each case optionally mono- or polysubstituted C₁-C₆-alkyl, where the substituents independently of one another may be selected from the group consisting of cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, CO₂H, C₂-C₆-alkoxycarbonyl, C₂-C₆-alkylcarbonyl, C₃-C₆-trialkylsilyl and optionally substituted phenyl, where the substituents independently of one another may be selected from the group consisting of one to three radicals W, C₁-C₆-haloalkyl, C₃-C₆-cycloalkyl and phenyl or pyridyl, each of which is optionally mono- to trisubstituted by W,
M represents in each case an optionally mono- to tetrasubstituted ring which, in addition to the nitrogen atom attached to the substituent pair R¹³ and R¹⁸, (R¹⁵)₂ or (R¹⁶)₂ contains two to six carbon atoms and optionally additionally a further nitrogen, sulphur or oxygen atom and where the substituents independently of one another may be selected from the group consisting of C₁-C₂-alkyl, halogen, cyano, nitro and C₁-C₂-alkoxy,
W in each case independently represents C₁-C₄-alkyl, C₂-C₄-alkenyl, C₂-C₄-alkynyl, C₃-C₆-cycloalkyl, C₁-C₄-haloalkyl, C₂-C₄-haloalkenyl, C₂-C₄-haloalkynyl, C₃-C₆-halocycloalkyl, halogen, cyano, nitro, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylamino, C₂-C₈-dialkylamino, C₃-C₆-cycloalkylamino, (C₁-C₄-alkyl)C₃-C₆-cycloalkylamino, C₂-C₄-alkylcarbonyl, C₂-C₆-alkoxycarbonyl, CO₂H, C₂-C₆-alkylaminocarbonyl, C₃-C₈-dialkylaminocarbonyl or C₃-C₆-trialkylsilyl,
n in each case independently represents 0 or 1,
p in each case independently represents 0, 1 or 2.

If (a) R⁵ represents hydrogen, C₁-C₆-alkyl, C₁-C₆-haloalkyl, C₂-C₆-haloalkenyl, C₂-C₆-haloalkynyl, C₁-C₄-haloalkoxy, C₁-C₄-haloalkylthio or halogen and (b) R⁸ represents hydrogen, C₁-C₆-alkyl, C₁-C₆-haloalkyl, C₂-C₆-haloalkenyl, C₂-C₆-haloalkynyl, C₁-C₄-haloalkoxy, C₁-C₄-haloalkylthio, halogen, C₂-C₄-alkylcarbonyl, C₂-C₆-alkoxycarbonyl, C₂-C₆-alkylaminocarbonyl or C₃-C₈-dialkylaminocarbonyl, then (c) at least one substituent selected from the group consisting of R⁶, R¹¹ and R¹² is present and (d), if R¹² is not present, at least one R⁶ or R¹¹ is different from C₂-C₆-alkylcarbonyl, C₂-C₆ alkoxycarbonyl, C₂-C₆-alkylaminocarbonyl and C₃-C₈-dialkylaminocarbonyl.

The compounds according to the general formula (I) include N-oxides and salts.

Depending inter alia on the nature of the substituents, the compounds of the formula (I) can be present as geometrical and/or optical isomers or isomer mixtures of varying composition which, if appropriate, can be separated in a customary manner. The present invention provides both the pure isomers and the isomer mixtures, their preparation and use and compositions comprising them. However, for the sake of simplicity, hereinbelow only compounds of the formula (I) are referred to, although what is meant is both the pure compounds and, if appropriate, mixtures having various proportions of isomeric compounds.

Preference is given to compounds of the formula (I-1)

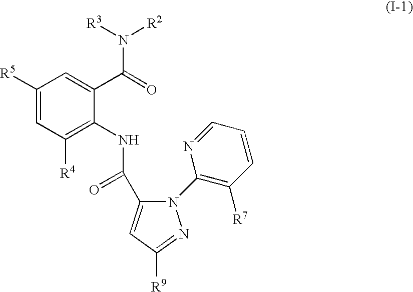

in which
R² represents hydrogen or C₁-C₆-alkyl,
R³ represents C₁-C₆-alkyl which is optionally substituted by one R⁶,
R⁴ represents C₁-C₄-alkyl, C₁-C₂-haloalkyl, C₁-C₂-haloalkoxy or halogen,
R⁵ represents hydrogen, C₁-C₄-alkyl, C₁-C₂-haloalkyl, C₁-C₂-haloalkoxy, cyano or halogen,
R⁶ represents —C(=E²)R¹⁹, -LC(=E²)R¹⁹, —C(=E²)LR¹⁹ or -LC(=E²)LR¹⁹, where each E² independently represents O, S, N—R¹⁵, N—OR¹⁵, N—N(R⁵)₂, and each L independently represents O or NR¹⁸,
R⁷ represents C₁-C₄-haloalkyl or halogen,
R⁹ represents C₁-C₂-haloalkyl, C₁-C₂-haloalkoxy, S(O)ₚC₁-C₂-haloalkyl or halogen,
R¹⁵ in each case independently represents hydrogen or represents in each case optionally substituted C₁-C₆-haloalkyl or C₁-C₆-alkyl, where the substituents independently of one another may be selected from the group consisting of cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl and C₁-C₄-haloalkylsulphonyl,
R¹⁸ represents in each case hydrogen or C₁-C₄-alkyl,
R¹⁹ in each case independently represents hydrogen or C₁-C₆-alkyl,
p independently represents 0, 1, 2.

In the radical definitions mentioned as being preferred, halogen represents fluorine, chlorine, bromine and iodine, in particular fluorine, chlorine and bromine.

Particular preference is given to compounds of the formula (I-1) in which
R² represents hydrogen or methyl,
R³ represents C₁-C₄-alkyl (in particular methyl, ethyl, n-, isopropyl, n-, iso-, sec-, tert-butyl),
R⁴ represents methyl, trifluoromethyl, trifluoromethoxy, fluorine, chlorine, bromine or iodine,
R⁵ represents hydrogen, cyano, fluorine, chlorine, bromine, iodine, trifluoromethyl or trifluoromethoxy,
R⁷ represents chlorine or bromine,
R⁹ represents trifluoromethyl, chlorine, bromine, difluoromethoxy or trifluoroethoxy.

Very particular preference is given to the following compounds of the formula

| | |
|---|---|
| | (I-1) |
| 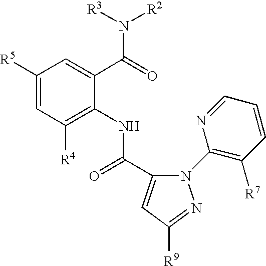 | |

| Example | | | | | | | |
|---|---|---|---|---|---|---|---|
| No. | R² | R³ | R⁴ | R⁵ | R⁷ | R⁹ | m.p.(° C.) |
| I-1-1 | H | Me | Me | Cl | Cl | CF₃ | 185-186 |
| I-1-2 | H | Me | Me | Cl | Cl | Cl | 225-226 |
| I-1-3 | H | Me | Me | Cl | Cl | Br | 162-164 |
| I-1-4 | H | i-Pr | Me | Cl | Cl | CF₃ | 195-196 |
| I-1-5 | H | i-Pr | Me | Cl | Cl | Cl | 173-175 |
| I-1-6 | H | i-Pr | Me | Cl | Cl | Br | 159-161 |
| I-1-7 | H | Me | Me | Br | Cl | CF₃ | 222-223 |
| I-1-8 | H | i-Pr | Me | Br | Cl | CF₃ | 197-198 |
| I-1-9 | H | Me | Me | Br | Cl | Cl | 140-141 |
| I-1-10 | H | i-Pr | Me | Br | Cl | Cl | 152-153 |
| I-1-11 | H | Me | Me | Br | Cl | Br | 147-149 |
| I-1-12 | H | i-Pr | Me | Br | Cl | Br | 185-187 |
| I-1-13 | H | Me | Me | I | Cl | CF₃ | 199-200 |
| I-1-14 | H | i-Pr | Me | I | Cl | CF₃ | 188-189 |
| I-1-15 | H | Me | Me | I | Cl | Cl | 233-234 |
| I-1-16 | H | i-Pr | Me | I | Cl | Cl | 189-190 |
| I-1-17 | H | Me | Me | I | Cl | Br | 229-230 |
| I-1-18 | H | i-Pr | Me | I | Cl | Br | 191-192 |
| I-1-19 | H | i-Pr | Me | CN | Cl | CF₃ | >250 |
| I-1-20 | H | Me | Me | CN | Cl | CF₃ | 214-216 |
| I-1-21 | H | i-Pr | Me | CN | Cl | Br | * |
| I-1-22 | H | Me | Me | CN | Cl | Br | * |
| I-1-23 | H | i-Pr | Me | CN | Cl | Cl | * |
| I-1-24 | H | Me | Me | CN | Cl | Cl | * |

* = 1H-NMR data (CDCl₃):

I-1-80: 10.60 (s, 1H), 8.47 (s, 1H), 7.85 (dd, 1H), 7.56 (s, 2H), 7.39 (dd, 1H), 7.06 (s, 1H), 6.04 (b d, 1H), 4.20 (m, 1H), 2.24 (s, 3H), 1.26 (s, 6H).
I-1-81: 10.55 (s, 1H), 8.45 (s, 1H), 7.85 (dd, 1H), 7.57 (m, 2H), 7.37 (dd, 1H), 7.05 (s, 1H), 6.30 (b q, 1H), 2.98 (d, 3H), 2.24 (s, 3H).
I-1-82: 10.12 (s, 1H), 8.56 (d, 1H), 7.85 (d, 1H), 7.58 (m, 2H), 7.40 (dd, 1H), 6.97 (s, 1H), 6.00 (b d, 1H), 4.22 (m, 1H), 2.25 (s, 3H), 1.26 (d, 6H).
I-1-83: 10.55 (s, 1H), 8.45 (s, 1H), 7.85 (dd, 1H), 7.55 (s, 2H), 7.40 (dd, 1H), 6.97 (s, 1H), 6.30 (b q, 1H), 2.98 (d, 3H), 2.24 (s, 3H).

Especially preferred are the following compounds:

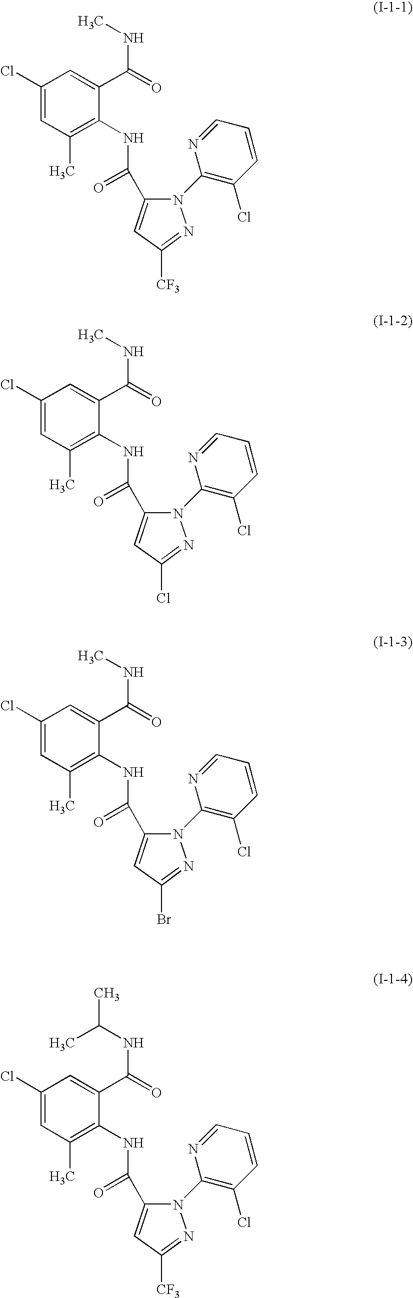

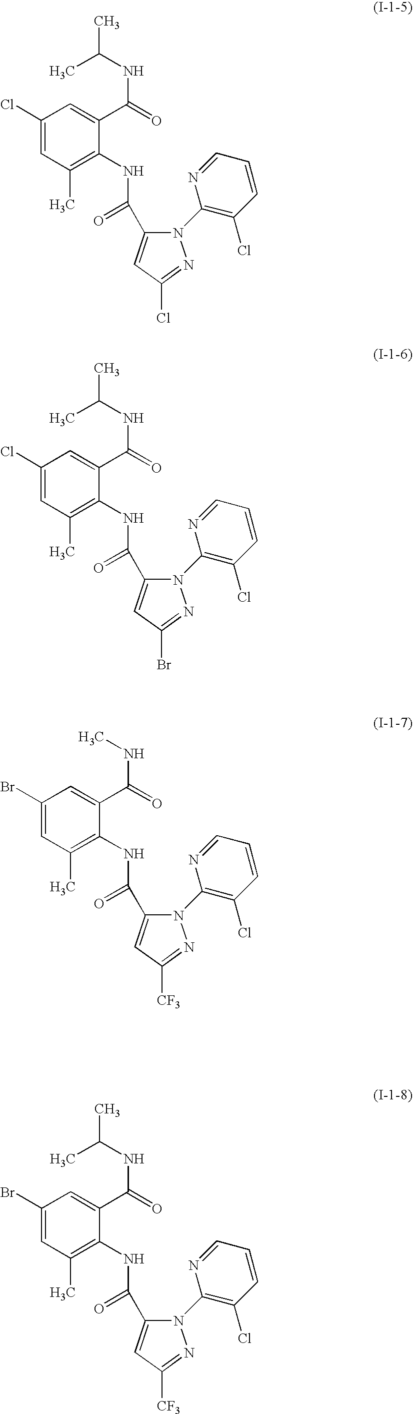

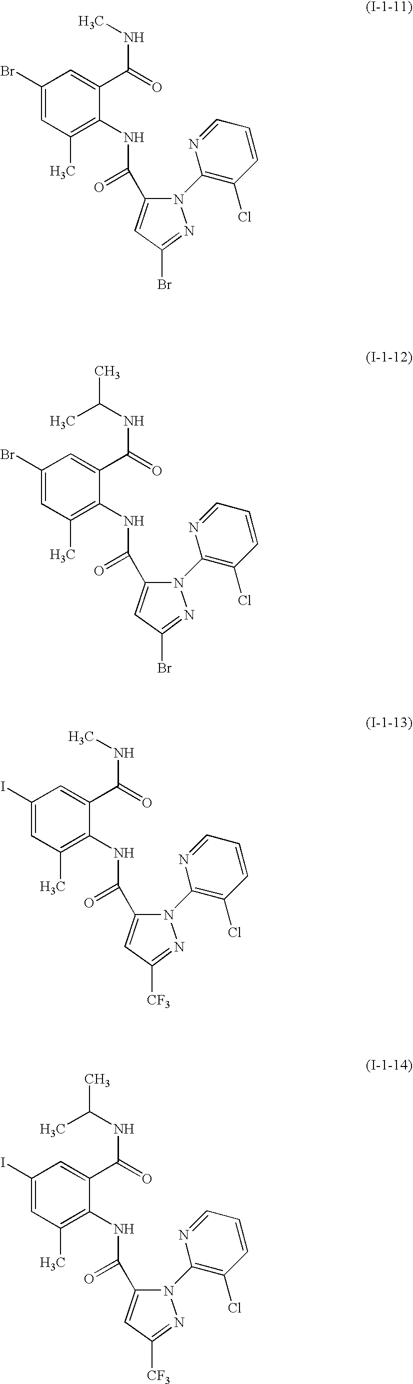

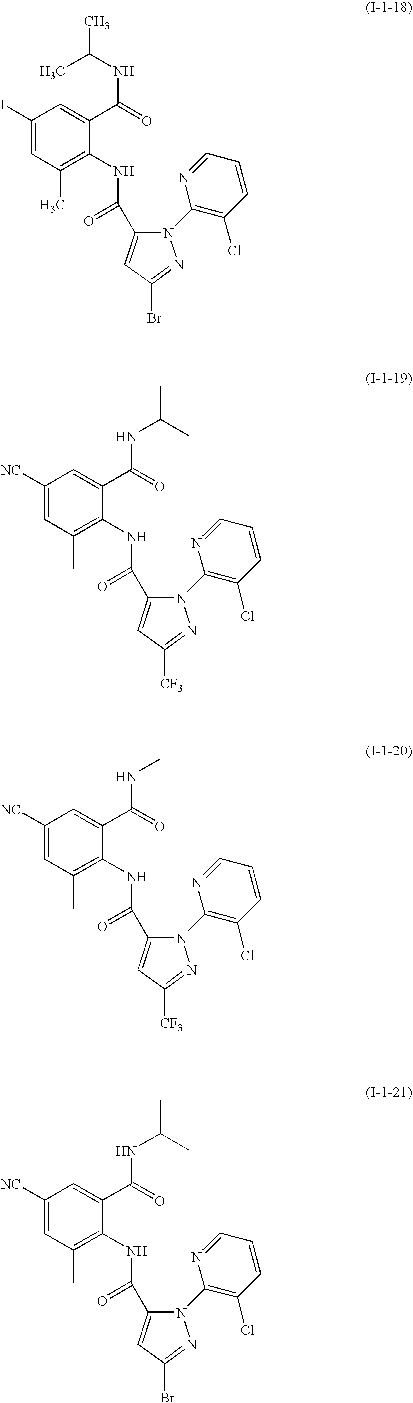

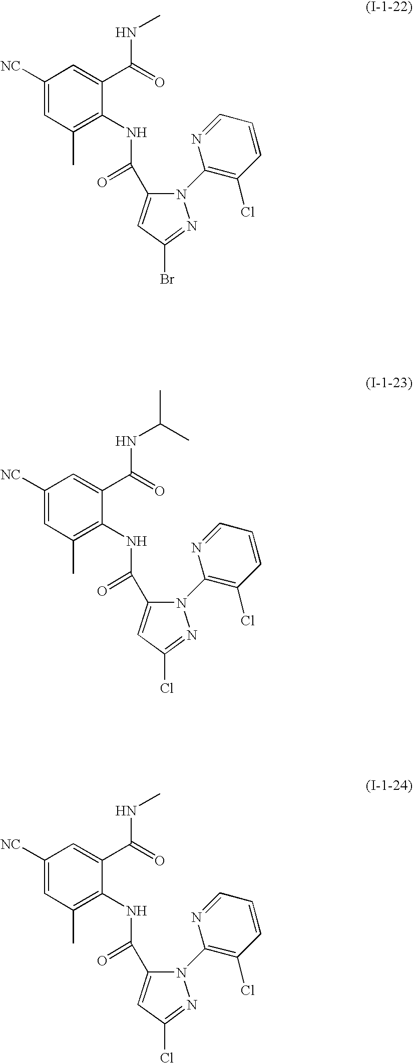

Phthalic acid diamides have a broad insecticidal action; however, in individual cases the activity is unsatisfactory.

Descriptions have already been given in the literature to the effect that the activity of various active compounds can be increased through addition of ammonium or phosphonium salts. The salts in question, however, are salts with a detergent effect (for example WO 95/017817) and/or salts having relatively long alkyl and/or aryl substituents, which have a permeabilizing effect or which increase the solubility of the active compound (for example EP-A 0 453 086, EP-A 0 664 081, FR-A 2 600 494, U.S. Pat. No. 4,844,734, U.S. Pat. No. 5,462,912, U.S. Pat. No. 5,538,937, Ser. No. 03/0224939, Ser. No. 05/0009880, Ser. No. 05/0096386). Furthermore, the prior art describes the activity only for certain active compounds and/or certain applications of the composition in question. In still other cases, they are salts of sulphonic acids where the acids for their part have a paralysing action on insects (U.S. Pat. No. 2,842,476). An activity increase through ammonium sulphate is described for the herbicides glyphosate and phosphinothricin (U.S. Pat. No. 6,645,914, EP-A 0 036 106). However, this prior art neither discloses nor suggests a corresponding activity for insecticides.

Also, the use of ammonium sulphate as a formulating auxiliary has been described for certain active compounds and applications (WO 92/16108), but it is used there for the purpose of stabilizing the formulation, not for increasing activity.

It has now been found, entirely surprisingly, that the activity of insecticides from the class of the anthranilic acid diamides can be increased significantly through the addition of ammonium salts and/or phosphonium salts to the as-used solution (tank mix application) or through the incorporation of these salts into a formulation comprising such insecticides. Accordingly, the present invention provides the use of ammonium salts and/or phosphonium salts for increasing the activity of crop protection compositions comprising insecticidally active inhibitors of the nicotinic acetylcholine receptor as active compound. The invention also provides compositions comprising such insecticides and activity-increasing ammonium salts and/or phosphonium salts, specifically including not only formulated active compounds but also ready-to-use compositions (spray liquors). Finally, the invention also provides the use of these compositions for controlling harmful insects.

Ammonium salts and phosphonium salts which, according to the invention, increase the activity of crop protection compositions comprising anthranilic acid diamides are defined by formula (II)

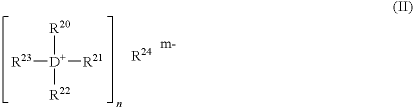

in which
D represents nitrogen or phosphorus,
D preferably represents nitrogen,
R²⁰, R²¹, R²² and R²³ independently of one another represent hydrogen or in each case optionally substituted C₁-C₈-alkyl or mono- or polyunsaturated, optionally substituted C₁-C₈-alkylene, where the substituents may be selected from the group consisting of halogen, nitro and cyano,
R²⁰, R²¹, R²² and R²³ preferably independently of one another represent hydrogen or in each case optionally substituted C₁-C₄-alkyl, where the substituents may be selected from the group consisting of halogen, nitro and cyano,
R²⁰, R²¹, R²² and R²³ particularly preferably independently of one another represent hydrogen, methyl, ethyl, n-propyl, isopropyl, n-butyl, isobutyl, s-butyl or t-butyl,
R²⁰, R²¹, R²² and R²³ very particularly preferably represent hydrogen,
R²⁰, R²¹, R²² and R²³ furthermore very particularly preferably both represent methyl or both represent ethyl,
n represents 1, 2, 3 or 4,
n preferably represents 1 or 2,
R²⁴ represents an inorganic or organic anion,
R²⁴ preferably represents bicarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate, citrate or oxalate,
R²⁴ furthermore preferably represents carbonate, pentaborate, sulphite, benzoate, hydrogenoxalate, hydrogencitrate, methylsulphate or tetrafluoroborate,
R²⁴ particularly preferably represents lactate, sulphate, nitrate, thiosulphate, thiocyanate, citrate, oxalate or formate,
R²⁴ furthermore particularly preferably represents monohydrogenphosphate or dihydrogenphosphate and
R²⁴ very particularly preferably represents sulphate.

The ammonium salts and phosphonium salts of the formula (II) can be used in a broad concentration range to increase the activity of crop protection compositions comprising anthranilamides. In general, the ammonium salts or phosphonium salts are used in the ready-to-use crop protection composition in a concentration of from 0.5 to 80 mmol/l, preferably from 0.75 to 37.5 mmol/l, particularly preferably from 1.5 to 25 mmol/l. In the case of a formulated product, the ammonium salt concentration and/or phosphonium salt concentration in the formulation is chosen such that it is within these stated general, preferred or very preferred ranges after the formulation has been diluted to the desired active compound concentration. The concentration of the salt in the formulation here is typically 1-50% by weight.

In a preferred embodiment of the invention, the activity is increased by adding to the crop protection compositions not only an ammonium salt and/or phosphonium salt but also, additionally, a penetrant. It is considered entirely surprising that even in these cases an even greater activity increase is observed. The present invention therefore likewise provides the use of a combination of penetrant and ammonium salts and/or phosphonium salts for increasing the activity of crop protection compositions comprising insecticidally active inhibitors of the nicotinic acetylcholine receptor as active compound. The invention likewise provides compositions which comprise insecticidally active inhibitors of the nicotinic acetylcholine receptor, penetrants and ammonium salts and/or phosphonium salts, including specifically not only formulated active compounds but also ready-to-use compositions (spray liquors). The invention additionally provides, finally, for the use of these compositions for controlling harmful insects.

Suitable penetrants in the present context include all those substances which are typically used to enhance the penetration of agrochemically active compounds into plants. Penetrants are defined in this context by their ability to penetrate from the aqueous spray liquor and/or from the spray coating into the cuticle of the plant and thereby to increase the mobility of active compounds in the cuticle. The method described in the literature (Baur et al., 1997, Pesticide Science 51, 131-152) can be used in order to determine this property.

Suitable penetrants are, for example, alkanol alkoxylates. Penetrants according to the invention are alkanol alkoxylates of the formula

R—O-(-AO)_{V}—R′ (III)

in which
R represents straight-chain or branched alkyl having 4 to 20 carbon atoms,
R′ represents hydrogen, methyl, ethyl, n-propyl, isopropyl, n-butyl, isobutyl, t-butyl, n-pentyl or n-hexyl,
AO represents an ethylene oxide radical, a propylene oxide radical, a butylene oxide radical or represents mixtures of ethylene oxide and propylene oxide radicals or butylene oxide radicals and
v represents numbers from 2 to 30.

A preferred group of penetrants are alkanol alkoxylates of the formula

R—O-(-EO—)_{w}—R′ (III-a)

in which
R is as defined above,
R′ is as defined above,
EO represents —CH₂—CH₂—O— and
w represents numbers from 2 to 20.

A further preferred group of penetrants are alkanol alkoxylates of the formula

R—O-(-EO—)_{b}—(—PO—)_{q}—R′ (III-b)

in which
R is as defined above,
R′ is as defined above,
EO represents —CH₂—CH₂—O—,
PO represents

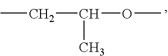

b represents numbers from 1 to 10 and
q represents numbers from 1 to 10.

A further preferred group of penetrants are alkanol alkoxylates of the formula

R—O—(—PO—)ᵣ-(EO—)ₛ—R (III-c)

in which
R is as defined above,
R′ is as defined above,
EO represents —CH₂—CH₂—O—,
PO represents

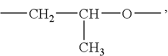

r represents numbers from 1 to 10 and
s represents numbers from 1 to 10.

A further preferred group of penetrants are alkanol alkoxylates of the formula

R—O-(-EO—)_{b}—(—BO—)_{q}—R′ (III-d)

in which
R and R′ are as defined above,
EO represents CH₂—CH₂—O—,
BO represents

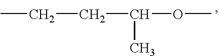

b represents numbers from 1 to 10 and
q represents numbers from 1 to 10.

A further preferred group of penetrants are alkanol alkoxylates of the formula

R—O—(—BO—)ᵣ-(-EO—)ₛ—R′ (III-e)

in which
R and R′ are as defined above,
BO represents

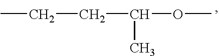

EO represents CH₂—CH₂—O—,
r represents numbers from 1 to 10 and
s represents numbers from 1 to 10.

A further preferred group of penetrants are alkanol alkoxylates of the formula

CH₃—(CH₂)ᵣ—CH₂—O—(—CH₂—CH₂—O—)ᵤ—R′ (III-f)

in which
R′ is as defined above,
t represents numbers from 8 to 13 and
u represents numbers from 6 to 17.

In the formulae given above,
R preferably represents butyl, isobutyl, n-pentyl, isopentyl, neopentyl, n-hexyl, isohexyl, n-octyl, isooctyl, 2-ethylhexyl, nonyl, isononyl, decyl, n-dodecyl, isododecyl, lauryl, myristyl, isotridecyl, trimethylnonyl, palmityl, stearyl or eicosyl.

An example which may be mentioned of an alkanol alkoxylate of the formula (III-c) is 2-ethylhexyl alkoxylate of the formula

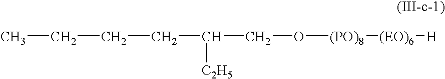

in which
EO represents —CH₂—CH₂—O—,
PO represents

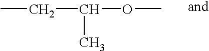

the numbers 8 and 6 are average values.

An example which may be mentioned of an alkanol alkoxylate of the formula (III-d) is the formula

CH₃—(CH₂)₁₀—O-(-EO—)₆—(—BO—)₂—CH₃ (III-d-1)

in which
EO represents CH₂—CH₂—O—,
BO represents

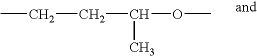

the numbers 10, 6 and 2 are average values.

Particularly preferred alkanol alkoxylates of the formula (I-f) are compounds of this formula in which
t represents numbers from 9 to 12 and
u represents numbers from 7 to 9.

The alkanol alkoxylate of the formula (III-f-1)

CH₃—(CH₂)ₜ—CH₂—O-(—CH₂—CH₂—O—)ᵤ—H (III-f-1)

in which
t represents the average value 10.5 and
u represents the average value 8.4
   may be mentioned as being very particularly preferred.

The above formulae provide a general definition of the alkanol alkoxylates. These substances are mixtures of substances of the stated type with different chain lengths. The indices therefore have average values which may also deviate from whole numbers.

The alkanol alkoxylates of the formulae stated are known and in some cases are available commercially or can be prepared by known methods (cf. WO 98-35 553, WO 00-35 278 and EP-A 0 681 865).

Suitable penetrants also include, for example, substances which promote the solubility of the compounds of the formula (I) in a spray coating. These include, for example, mineral and vegetable oils. Suitable oils are all mineral or vegetable oils—modified or otherwise—which can typically be used in agrochemical compositions. Mention may be made by way of example of sunflower oil, rapeseed oil, olive oil, castor oil, colza oil, maizeseed oil, cottonseed oil and soybean oil, or the esters of said oils. Preference is given to rapeseed oil, sunflower oil and their methyl or ethyl esters.

The concentration of penetrants in the compositions according to the invention can be varied within a wide range. In the case of a formulated crop protection composition, it is generally from 1 to 95% by weight, preferably from 1 to 55% by weight, particularly preferably from 15 to 40% by weight. In the ready-to-use compositions (spray liquors), the concentration is generally between 0.1 and 10 g/l, preferably between 0.5 and 5 g/l.

Inventively emphasized combinations of active compound, salt and penetrant are listed in the table below. Here, “as per test” means that any compound that acts as a penetrant in the cuticle penetration test (Baur et al., 1997, Pesticide Science 51, 131-152) is suitable.

| | Active | | |
|---|---|---|---|
| | com- | | |
| # | pound | Salt | Penetrant |
| 1 | (I-1-1) | ammonium sulphate | as per test |
| 2 | (I-1-1) | ammonium lactate | as per test |
| 3 | (I-1-1) | ammonium nitrate | as per test |
| 4 | (I-1-1) | ammonium thiosulphate | as per test |
| 5 | (I-1-1) | ammonium thiocyanate | as per test |
| 6 | (I-1-1) | ammonium citrate | as per test |
| 7 | (I-1-1) | ammonium oxalate | as per test |
| 8 | (I-1-1) | ammonium formate | as per test |
| 9 | (I-1-1) | ammonium hydrogenphosphate | as per test |
| 10 | (I-1-1) | ammonium dihydrogenphosphate | as per test |
| 11 | (I-1-1) | ammonium carbonate | as per test |
| 12 | (I-1-1) | ammonium benzoate | as per test |
| 13 | (I-1-1) | ammonium sulphite | as per test |
| 14 | (I-1-1) | ammonium benzoate | as per test |
| 15 | (I-1-1) | ammonium hydrogenoxalate | as per test |
| 16 | (I-1-1) | ammonium hydrogencitrate | as per test |
| 17 | (I-1-1) | tetramethylammonium sulphate | as per test |
| 18 | (I-1-1) | tetramethylammonium lactate | as per test |
| 19 | (I-1-1) | tetramethylammonium nitrate | as per test |
| 20 | (I-1-1) | tetramethylammonium thiosulphate | as per test |
| 21 | (I-1-1) | tetramethylammonium thiocyanate | as per test |
| 22 | (I-1-1) | tetramethylammonium citrate | as per test |
| 23 | (I-1-1) | tetramethylammonium oxalate | as per test |
| 24 | (I-1-1) | tetramethylammonium formate | as per test |
| 25 | (I-1-1) | tetramethylammonium hydrogenphosphate | as per test |
| 26 | (I-1-1) | tetramethylammonium dihydrogenphosphate | as per test |
| 27 | (I-1-1) | tetraethylammonium sulphate | as per test |
| 28 | (I-1-1) | tetraethylammonium lactate | as per test |
| 29 | (I-1-1) | tetraethylammonium nitrate | as per test |
| 30 | (I-1-1) | tetraethylammonium thiosulphate | as per test |
| 31 | (I-1-1) | tetraethylammonium thiocyanate | as per test |
| 32 | (I-1-1) | tetraethylammonium citrate | as per test |
| 33 | (I-1-1) | tetraethylammonium oxalate | as per test |
| 34 | (I-1-1) | tetraethylammonium formate | as per test |
| 35 | (I-1-1) | tetraethylammonium hydrogenphosphate | as per test |
| 36 | (I-1-1) | tetraethylammonium dihydrogenphosphate | as per test |
| 37 | (I-1-2) | ammonium sulphate | as per test |
| 38 | (I-1-2) | ammonium lactate | as per test |
| 39 | (I-1-2) | ammonium nitrate | as per test |
| 40 | (I-1-2) | ammonium thiosulphate | as per test |
| 41 | (I-1-2) | ammonium thiocyanate | as per test |
| 42 | (I-1-2) | ammonium citrate | as per test |
| 43 | (I-1-2) | ammonium oxalate | as per test |
| 44 | (I-1-2) | ammonium formate | as per test |
| 45 | (I-1-2) | ammonium hydrogenphosphate | as per test |
| 46 | (I-1-2) | ammonium dihydrogenphosphate | as per test |
| 47 | (I-1-2) | ammonium carbonate | as per test |
| 48 | (I-1-2) | ammonium benzoate | as per test |
| 49 | (I-1-2) | ammonium sulphite | as per test |
| 50 | (I-1-2) | ammonium benzoate | as per test |
| 51 | (I-1-2) | ammonium hydrogenoxalate | as per test |
| 52 | (I-1-2) | ammonium hydrogencitrate | as per test |
| 53 | (I-1-2) | tetramethylammonium sulphate | as per test |
| 54 | (I-1-2) | tetramethylammonium lactate | as per test |
| 55 | (I-1-2) | tetramethylammonium nitrate | as per test |
| 56 | (I-1-2) | tetramethylammonium thiosulphate | as per test |
| 57 | (I-1-2) | tetramethylammonium thiocyanate | as per test |
| 58 | (I-1-2) | tetramethylammonium citrate | as per test |
| 59 | (I-1-2) | tetramethylammonium oxalate | as per test |
| 60 | (I-1-2) | tetramethylammonium formate | as per test |
| 61 | (I-1-2) | tetramethylammonium hydrogenphosphate | as per test |
| 62 | (I-1-2) | tetramethylammonium dihydrogenphosphate | as per test |
| 63 | (I-1-2) | tetraethylammonium sulphate | as per test |
| 64 | (I-1-2) | tetraethylammonium lactate | as per test |
| 65 | (I-1-2) | tetraethylammonium nitrate | as per test |
| 66 | (I-1-2) | tetraethylammonium thiosulphate | as per test |
| 67 | (I-1-2) | tetraethylammonium thiocyanate | as per test |
| 68 | (I-1-2) | tetraethylammonium citrate | as per test |
| 69 | (I-1-2) | tetraethylammonium oxalate | as per test |
| 70 | (I-1-2) | tetraethylammonium formate | as per test |
| 71 | (I-1-2) | tetraethylammonium hydrogenphosphate | as per test |
| 72 | (I-1-2) | tetraethylammonium dihydrogenphosphate | as per test |
| 73 | (I-1-3) | ammonium sulphate | as per test |
| 74 | (I-1-3) | ammonium lactate | as per test |
| 75 | (I-1-3) | ammonium nitrate | as per test |
| 76 | (I-1-3) | ammonium thiosulphate | as per test |
| 77 | (I-1-3) | ammonium thiocyanate | as per test |
| 78 | (I-1-3) | ammonium citrate | as per test |
| 79 | (I-1-3) | ammonium oxalate | as per test |
| 80 | (I-1-3) | ammonium formate | as per test |
| 81 | (I-1-3) | ammonium hydrogenphosphate | as per test |
| 82 | (I-1-3) | ammonium dihydrogenphosphate | as per test |
| 83 | (I-1-3) | ammonium carbonate | as per test |
| 84 | (I-1-3) | ammonium benzoate | as per test |
| 85 | (I-1-3) | ammonium sulphite | as per test |
| 86 | (I-1-3) | ammonium benzoate | as per test |
| 87 | (I-1-3) | ammonium hydrogenoxalate | as per test |
| 88 | (I-1-3) | ammonium hydrogencitrate | as per test |
| 89 | (I-1-3) | tetramethylammonium sulphate | as per test |
| 90 | (I-1-3) | tetramethylammonium lactate | as per test |
| 91 | (I-1-3) | tetramethylammonium nitrate | as per test |
| 92 | (I-1-3) | tetramethylammonium thiosulphate | as per test |
| 93 | (I-1-3) | tetramethylammonium thiocyanate | as per test |
| 94 | (I-1-3) | tetramethylammonium citrate | as per test |
| 95 | (I-1-3) | tetramethylammonium oxalate | as per test |
| 96 | (I-1-3) | tetramethylammonium formate | as per test |
| 97 | (I-1-3) | tetramethylammonium hydrogenphosphate | as per test |
| 98 | (I-1-3) | tetramethylammonium dihydrogenphosphate | as per test |
| 99 | (I-1-3) | tetraethylammonium sulphate | as per test |
| 100 | (I-1-3) | tetraethylammonium lactate | as per test |
| 101 | (I-1-3) | tetraethylammonium nitrate | as per test |
| 102 | (I-1-3) | tetraethylammonium thiosulphate | as per test |
| 103 | (I-1-3) | tetraethylammonium thiocyanate | as per test |
| 104 | (I-1-3) | tetraethylammonium citrate | as per test |
| 105 | (I-1-3) | tetraethylammonium oxalate | as per test |
| 106 | (I-1-3) | tetraethylammonium formate | as per test |
| 107 | (I-1-3) | tetraethylammonium hydrogenphosphate | as per test |
| 108 | (I-1-3) | tetraethylammonium dihydrogenphosphate | as per test |
| 109 | (I-1-4) | ammonium sulphate | as per test |
| 110 | (I-1-4) | ammonium lactate | as per test |
| 111 | (I-1-4) | ammonium nitrate | as per test |
| 112 | (I-1-4) | ammonium thiosulphate | as per test |
| 113 | (I-1-4) | ammonium thiocyanate | as per test |
| 114 | (I-1-4) | ammonium citrate | as per test |
| 115 | (I-1-4) | ammonium oxalate | as per test |
| 116 | (I-1-4) | ammonium formate | as per test |
| 117 | (I-1-4) | ammonium hydrogenphosphate | as per test |
| 118 | (I-1-4) | ammonium dihydrogenphosphate | as per test |
| 119 | (I-1-4) | ammonium carbonate | as per test |
| 120 | (I-1-4) | ammonium benzoate | as per test |
| 121 | (I-1-4) | ammonium sulphite | as per test |
| 122 | (I-1-4) | ammonium benzoate | as per test |
| 123 | (I-1-4) | ammonium hydrogenoxalate | as per test |
| 124 | (I-1-4) | ammonium hydrogencitrate | as per test |
| 125 | (I-1-4) | tetramethylammonium sulphate | as per test |
| 126 | (I-1-4) | tetramethylammonium lactate | as per test |
| 127 | (I-1-4) | tetramethylammonium nitrate | as per test |
| 128 | (I-1-4) | tetramethylammonium thiosulphate | as per test |
| 129 | (I-1-4) | tetramethylammonium thiocyanate | as per test |
| 130 | (I-1-4) | tetramethylammonium citrate | as per test |
| 131 | (I-1-4) | tetramethylammonium oxalate | as per test |
| 132 | (I-1-4) | tetramethylammonium formate | as per test |
| 133 | (I-1-4) | tetramethylammonium hydrogenphosphate | as per test |
| 134 | (I-1-4) | tetramethylammonium dihydrogenphosphate | as per test |
| 135 | (I-1-4) | tetraethylammonium sulphate | as per test |
| 136 | (I-1-4) | tetraethylammonium lactate | as per test |
| 137 | (I-1-4) | tetraethylammonium nitrate | as per test |
| 138 | (I-1-4) | tetraethylammonium thiosulphate | as per test |
| 139 | (I-1-4) | tetraethylammonium thiocyanate | as per test |
| 140 | (I-1-4) | tetraethylammonium citrate | as per test |
| 141 | (I-1-4) | tetraethylammonium oxalate | as per test |
| 142 | (I-1-4) | tetraethylammonium formate | as per test |
| 143 | (I-1-4) | tetraethylammonium hydrogenphosphate | as per test |
| 144 | (I-1-4) | tetraethylammonium dihydrogenphosphate | as per test |
| 145 | (I-1-5) | ammonium sulphate | as per test |
| 146 | (I-1-5) | ammonium lactate | as per test |
| 147 | (I-1-5) | ammonium nitrate | as per test |
| 148 | (I-1-5) | ammonium thiosulphate | as per test |
| 149 | (I-1-5) | ammonium thiocyanate | as per test |
| 150 | (I-1-5) | ammonium citrate | as per test |
| 151 | (I-1-5) | ammonium oxalate | as per test |
| 152 | (I-1-5) | ammonium formate | as per test |
| 153 | (I-1-5) | ammonium hydrogenphosphate | as per test |
| 154 | (I-1-5) | ammonium dihydrogenphosphate | as per test |
| 155 | (I-1-5) | ammonium carbonate | as per test |
| 156 | (I-1-5) | ammonium benzoate | as per test |
| 157 | (I-1-5) | ammonium sulphite | as per test |
| 158 | (I-1-5) | ammonium benzoate | as per test |
| 159 | (I-1-5) | ammonium hydrogenoxalate | as per test |
| 160 | (I-1-5) | ammonium hydrogencitrate | as per test |
| 161 | (I-1-5) | tetramethylammonium sulphate | as per test |
| 162 | (I-1-5) | tetramethylammonium lactate | as per test |
| 163 | (I-1-5) | tetramethylammonium nitrate | as per test |
| 164 | (I-1-5) | tetramethylammonium thiosulphate | as per test |
| 165 | (I-1-5) | tetramethylammonium thiocyanate | as per test |
| 166 | (I-1-5) | tetramethylammonium citrate | as per test |
| 167 | (I-1-5) | tetramethylammonium oxalate | as per test |
| 168 | (I-1-5) | tetramethylammonium formate | as per test |
| 169 | (I-1-5) | tetramethylammonium hydrogenphosphate | as per test |
| 170 | (I-1-5) | tetramethylammonium dihydrogenphosphate | as per test |
| 171 | (I-1-5) | tetraethylammonium sulphate | as per test |
| 172 | (I-1-5) | tetraethylammonium lactate | as per test |
| 173 | (I-1-5) | tetraethylammonium nitrate | as per test |
| 174 | (I-1-5) | tetraethylammonium thiosulphate | as per test |
| 175 | (I-1-5) | tetraethylammonium thiocyanate | as per test |
| 176 | (I-1-5) | tetraethylammonium citrate | as per test |
| 177 | (I-1-5) | tetraethylammonium oxalate | as per test |
| 178 | (I-1-5) | tetraethylammonium formate | as per test |
| 179 | (I-1-5) | tetraethylammonium hydrogenphosphate | as per test |
| 180 | (I-1-5) | tetraethylammonium dihydrogenphosphate | as per test |
| 181 | (I-1-6) | ammonium sulphate | as per test |
| 182 | (I-1-6) | ammonium lactate | as per test |
| 183 | (I-1-6) | ammonium nitrate | as per test |
| 184 | (I-1-6) | ammonium thiosulphate | as per test |
| 185 | (I-1-6) | ammonium thiocyanate | as per test |
| 186 | (I-1-6) | ammonium citrate | as per test |
| 187 | (I-1-6) | ammonium oxalate | as per test |
| 188 | (I-1-6) | ammonium formate | as per test |
| 189 | (I-1-6) | ammonium hydrogenphosphate | as per test |
| 190 | (I-1-6) | ammonium dihydrogenphosphate | as per test |
| 191 | (I-1-6) | ammonium carbonate | as per test |
| 192 | (I-1-6) | ammonium benzoate | as per test |
| 193 | (I-1-6) | ammonium sulphite | as per test |
| 194 | (I-1-6) | ammonium benzoate | as per test |
| 195 | (I-1-6) | ammonium hydrogenoxalate | as per test |
| 196 | (I-1-6) | ammonium hydrogencitrate | as per test |
| 197 | (I-1-6) | tetramethylammonium sulphate | as per test |
| 198 | (I-1-6) | tetramethylammonium lactate | as per test |
| 199 | (I-1-6) | tetramethylammonium nitrate | as per test |
| 200 | (I-1-6) | tetramethylammonium thiosulphate | as per test |
| 201 | (I-1-6) | tetramethylammonium thiocyanate | as per test |
| 202 | (I-1-6) | tetramethylammonium citrate | as per test |
| 203 | (I-1-6) | tetramethylammonium oxalate | as per test |
| 204 | (I-1-6) | tetramethylammonium formate | as per test |
| 205 | (I-1-6) | tetramethylammonium hydrogenphosphate | as per test |
| 206 | (I-1-6) | tetramethylammonium dihydrogenphosphate | as per test |
| 207 | (I-1-6) | tetraethylammonium sulphate | as per test |
| 208 | (I-1-6) | tetraethylammonium lactate | as per test |
| 209 | (I-1-6) | tetraethylammonium nitrate | as per test |
| 210 | (I-1-6) | tetraethylammonium thiosulphate | as per test |
| 211 | (I-1-6) | tetraethylammonium thiocyanate | as per test |
| 212 | (I-1-6) | tetraethylammonium citrate | as per test |
| 213 | (I-1-6) | tetraethylammonium oxalate | as per test |
| 214 | (I-1-6) | tetraethylammonium formate | as per test |
| 215 | (I-1-6) | tetraethylammonium hydrogenphosphate | as per test |
| 216 | (I-1-6) | tetraethylammonium dihydrogenphosphate | as per test |
| 217 | (I-1-7) | ammonium sulphate | as per test |
| 218 | (I-1-7) | ammonium lactate | as per test |
| 219 | (I-1-7) | ammonium nitrate | as per test |
| 220 | (I-1-7) | ammonium thiosulphate | as per test |
| 221 | (I-1-7) | ammonium thiocyanate | as per test |
| 222 | (I-1-7) | ammonium citrate | as per test |
| 223 | (I-1-7) | ammonium oxalate | as per test |
| 224 | (I-1-7) | ammonium formate | as per test |
| 225 | (I-1-7) | ammonium hydrogenphosphate | as per test |
| 226 | (I-1-7) | ammonium dihydrogenphosphate | as per test |
| 227 | (I-1-7) | ammonium carbonate | as per test |
| 228 | (I-1-7) | ammonium benzoate | as per test |
| 229 | (I-1-7) | ammonium sulphite | as per test |
| 230 | (I-1-7) | ammonium benzoate | as per test |
| 231 | (I-1-7) | ammonium hydrogenoxalate | as per test |
| 232 | (I-1-7) | ammonium hydrogencitrate | as per test |
| 233 | (I-1-7) | tetramethylammonium sulphate | as per test |
| 234 | (I-1-7) | tetramethylammonium lactate | as per test |
| 235 | (I-1-7) | tetramethylammonium nitrate | as per test |
| 236 | (I-1-7) | tetramethylammonium thiosulphate | as per test |
| 237 | (I-1-7) | tetramethylammonium thiocyanate | as per test |
| 238 | (I-1-7) | tetramethylammonium citrate | as per test |
| 239 | (I-1-7) | tetramethylammonium oxalate | as per test |
| 240 | (I-1-7) | tetramethylammonium formate | as per test |
| 241 | (I-1-7) | tetramethylammonium hydrogenphosphate | as per test |
| 242 | (I-1-7) | tetramethylammonium dihydrogenphosphate | as per test |
| 243 | (I-1-7) | tetraethylammonium sulphate | as per test |
| 244 | (I-1-7) | tetraethylammonium lactate | as per test |
| 245 | (I-1-7) | tetraethylammonium nitrate | as per test |
| 246 | (I-1-7) | tetraethylammonium thiosulphate | as per test |
| 247 | (I-1-7) | tetraethylammonium thiocyanate | as per test |
| 248 | (I-1-7) | tetraethylammonium citrate | as per test |
| 249 | (I-1-7) | tetraethylammonium oxalate | as per test |
| 250 | (I-1-7) | tetraethylammonium formate | as per test |
| 251 | (I-1-7) | tetraethylammonium hydrogenphosphate | as per test |
| 252 | (I-1-7) | tetraethylammonium dihydrogenphosphate | as per test |
| 253 | (I-1-8) | ammonium sulphate | as per test |
| 254 | (I-1-8) | ammonium lactate | as per test |
| 255 | (I-1-8) | ammonium nitrate | as per test |
| 256 | (I-1-8) | ammonium thiosulphate | as per test |
| 257 | (I-1-8) | ammonium thiocyanate | as per test |
| 258 | (I-1-8) | ammonium citrate | as per test |
| 259 | (I-1-8) | ammonium oxalate | as per test |
| 260 | (I-1-8) | ammonium formate | as per test |
| 261 | (I-1-8) | ammonium hydrogenphosphate | as per test |
| 262 | (I-1-8) | ammonium dihydrogenphosphate | as per test |
| 263 | (I-1-8) | ammonium carbonate | as per test |
| 264 | (I-1-8) | ammonium benzoate | as per test |
| 265 | (I-1-8) | ammonium sulphite | as per test |
| 266 | (I-1-8) | ammonium benzoate | as per test |
| 267 | (I-1-8) | ammonium hydrogenoxalate | as per test |
| 268 | (I-1-8) | ammonium hydrogencitrate | as per test |
| 269 | (I-1-8) | tetramethylammonium sulphate | as per test |
| 270 | (I-1-8) | tetramethylammonium lactate | as per test |
| 271 | (I-1-8) | tetramethylammonium nitrate | as per test |
| 272 | (I-1-8) | tetramethylammonium thiosulphate | as per test |
| 273 | (I-1-8) | tetramethylammonium thiocyanate | as per test |
| 274 | (I-1-8) | tetramethylammonium citrate | as per test |
| 275 | (I-1-8) | tetramethylammonium oxalate | as per test |
| 276 | (I-1-8) | tetramethylammonium formate | as per test |
| 277 | (I-1-8) | tetramethylammonium hydrogenphosphate | as per test |
| 278 | (I-1-8) | tetramethylammonium dihydrogenphosphate | as per test |
| 279 | (I-1-8) | tetraethylammonium sulphate | as per test |
| 280 | (I-1-8) | tetraethylammonium lactate | as per test |
| 281 | (I-1-8) | tetraethylammonium nitrate | as per test |
| 282 | (I-1-8) | tetraethylammonium thiosulphate | as per test |
| 283 | (I-1-8) | tetraethylammonium thiocyanate | as per test |
| 284 | (I-1-8) | tetraethylammonium citrate | as per test |
| 285 | (I-1-8) | tetraethylammonium oxalate | as per test |
| 286 | (I-1-8) | tetraethylammonium formate | as per test |
| 287 | (I-1-8) | tetraethylammonium hydrogenphosphate | as per test |
| 288 | (I-1-8) | tetraethylammonium dihydrogenphosphate | as per test |
| 289 | (I-1-11) | ammonium sulphate | as per test |
| 290 | (I-1-11) | ammonium lactate | as per test |
| 291 | (I-1-11) | ammonium nitrate | as per test |
| 292 | (I-1-11) | ammonium thiosulphate | as per test |
| 293 | (I-1-11) | ammonium thiocyanate | as per test |
| 294 | (I-1-11) | ammonium citrate | as per test |
| 295 | (I-1-11) | ammonium oxalate | as per test |
| 296 | (I-1-11) | ammonium formate | as per test |
| 297 | (I-1-11) | ammonium hydrogenphosphate | as per test |
| 298 | (I-1-11) | ammonium dihydrogenphosphate | as per test |
| 299 | (I-1-11) | ammonium carbonate | as per test |
| 300 | (I-1-11) | ammonium benzoate | as per test |
| 301 | (I-1-11) | ammonium sulphite | as per test |
| 302 | (I-1-11) | ammonium benzoate | as per test |
| 303 | (I-1-11) | ammonium hydrogenoxalate | as per test |
| 304 | (I-1-11) | ammonium hydrogencitrate | as per test |
| 305 | (I-1-11) | tetramethylammonium sulphate | as per test |
| 306 | (I-1-11) | tetramethylammonium lactate | as per test |
| 307 | (I-1-11) | tetramethylammonium nitrate | as per test |
| 308 | (I-1-11) | tetramethylammonium thiosulphate | as per test |
| 309 | (I-1-11) | tetramethylammonium thiocyanate | as per test |
| 310 | (I-1-11) | tetramethylammonium citrate | as per test |
| 311 | (I-1-11) | tetramethylammonium oxalate | as per test |
| 312 | (I-1-11) | tetramethylammonium formate | as per test |
| 313 | (I-1-11) | tetramethylammonium hydrogenphosphate | as per test |
| 314 | (I-1-11) | tetramethylammonium dihydrogenphosphate | as per test |
| 315 | (I-1-11) | tetraethylammonium sulphate | as per test |
| 316 | (I-1-11) | tetraethylammonium lactate | as per test |
| 317 | (I-1-11) | tetraethylammonium nitrate | as per test |
| 318 | (I-1-11) | tetraethylammonium thiosulphate | as per test |
| 319 | (I-1-11) | tetraethylammonium thiocyanate | as per test |
| 320 | (I-1-11) | tetraethylammonium citrate | as per test |
| 321 | (I-1-11) | tetraethylammonium oxalate | as per test |
| 322 | (I-1-11) | tetraethylammonium formate | as per test |
| 323 | (I-1-11) | tetraethylammonium hydrogenphosphate | as per test |
| 324 | (I-1-11) | tetraethylammonium dihydrogenphosphate | as per test |
| 325 | (I-1-12) | ammonium sulphate | as per test |
| 326 | (I-1-12) | ammonium lactate | as per test |
| 327 | (I-1-12) | ammonium nitrate | as per test |
| 328 | (I-1-12) | ammonium thiosulphate | as per test |
| 329 | (I-1-12) | ammonium thiocyanate | as per test |
| 330 | (I-1-12) | ammonium citrate | as per test |
| 331 | (I-1-12) | ammonium oxalate | as per test |
| 332 | (I-1-12) | ammonium formate | as per test |
| 333 | (I-1-12) | ammonium hydrogenphosphate | as per test |
| 334 | (I-1-12) | ammonium dihydrogenphosphate | as per test |
| 335 | (I-1-12) | ammonium carbonate | as per test |
| 336 | (I-1-12) | ammonium benzoate | as per test |
| 337 | (I-1-12) | ammonium sulphite | as per test |
| 338 | (I-1-12) | ammonium benzoate | as per test |
| 339 | (I-1-12) | ammonium hydrogenoxalate | as per test |
| 340 | (I-1-12) | ammonium hydrogencitrate | as per test |
| 341 | (I-1-12) | tetramethylammonium sulphate | as per test |
| 342 | (I-1-12) | tetramethylammonium lactate | as per test |
| 343 | (I-1-12) | tetramethylammonium nitrate | as per test |
| 344 | (I-1-12) | tetramethylammonium thiosulphate | as per test |
| 345 | (I-1-12) | tetramethylammonium thiocyanate | as per test |
| 346 | (I-1-12) | tetramethylammonium citrate | as per test |
| 347 | (I-1-12) | tetramethylammonium oxalate | as per test |
| 348 | (I-1-12) | tetramethylammonium formate | as per test |
| 349 | (I-1-12) | tetramethylammonium hydrogenphosphate | as per test |
| 350 | (I-1-12) | tetramethylammonium dihydrogenphosphate | as per test |
| 351 | (I-1-12) | tetraethylammonium sulphate | as per test |
| 352 | (I-1-12) | tetraethylammonium lactate | as per test |
| 353 | (I-1-12) | tetraethylammonium nitrate | as per test |
| 354 | (I-1-12) | tetraethylammonium thiosulphate | as per test |
| 355 | (I-1-12) | tetraethylammonium thiocyanate | as per test |
| 356 | (I-1-12) | tetraethylammonium citrate | as per test |
| 357 | (I-1-12) | tetraethylammonium oxalate | as per test |
| 358 | (I-1-12) | tetraethylammonium formate | as per test |
| 359 | (I-1-12) | tetraethylammonium hydrogenphosphate | as per test |
| 360 | (I-1-12) | tetraethylammonium dihydrogenphosphate | as per test |
| 361 | (I-1-13) | ammonium sulphate | as per test |
| 362 | (I-1-13) | ammonium lactate | as per test |
| 363 | (I-1-13) | ammonium nitrate | as per test |
| 364 | (I-1-13) | ammonium thiosulphate | as per test |
| 365 | (I-1-13) | ammonium thiocyanate | as per test |
| 366 | (I-1-13) | ammonium citrate | as per test |
| 367 | (I-1-13) | ammonium oxalate | as per test |
| 368 | (I-1-13) | ammonium formate | as per test |
| 369 | (I-1-13) | ammonium hydrogenphosphate | as per test |
| 370 | (I-1-13) | ammonium dihydrogenphosphate | as per test |
| 371 | (I-1-13) | ammonium carbonate | as per test |
| 372 | (I-1-13) | ammonium benzoate | as per test |
| 373 | (I-1-13) | ammonium sulphite | as per test |
| 374 | (I-1-13) | ammonium benzoate | as per test |
| 375 | (I-1-13) | ammonium hydrogenoxalate | as per test |
| 376 | (I-1-13) | ammonium hydrogencitrate | as per test |
| 377 | (I-1-13) | tetramethylammonium sulphate | as per test |
| 378 | (I-1-13) | tetramethylammonium lactate | as per test |
| 379 | (I-1-13) | tetramethylammonium nitrate | as per test |
| 380 | (I-1-13) | tetramethylammonium thiosulphate | as per test |
| 381 | (I-1-13) | tetramethylammonium thiocyanate | as per test |
| 382 | (I-1-13) | tetramethylammonium citrate | as per test |
| 383 | (I-1-13) | tetramethylammonium oxalate | as per test |
| 384 | (I-1-13) | tetramethylammonium formate | as per test |
| 385 | (I-1-13) | tetramethylammonium hydrogenphosphate | as per test |
| 386 | (I-1-13) | tetramethylammonium dihydrogenphosphate | as per test |
| 387 | (I-1-13) | tetraethylammonium sulphate | as per test |
| 388 | (I-1-13) | tetraethylammonium lactate | as per test |
| 389 | (I-1-13) | tetraethylammonium nitrate | as per test |
| 390 | (I-1-13) | tetraethylammonium thiosulphate | as per test |
| 391 | (I-1-13) | tetraethylammonium thiocyanate | as per test |
| 392 | (I-1-13) | tetraethylammonium citrate | as per test |
| 393 | (I-1-13) | tetraethylammonium oxalate | as per test |
| 394 | (I-1-13) | tetraethylammonium formate | as per test |
| 395 | (I-1-13) | tetraethylammonium hydrogenphosphate | as per test |
| 396 | (I-1-13) | tetraethylammonium dihydrogenphosphate | as per test |
| 397 | (I-1-14) | ammonium sulphate | as per test |
| 398 | (I-1-14) | ammonium lactate | as per test |
| 399 | (I-1-14) | ammonium nitrate | as per test |
| 400 | (I-1-14) | ammonium thiosulphate | as per test |
| 401 | (I-1-14) | ammonium thiocyanate | as per test |
| 402 | (I-1-14) | ammonium citrate | as per test |
| 403 | (I-1-14) | ammonium oxalate | as per test |
| 404 | (I-1-14) | ammonium formate | as per test |
| 405 | (I-1-14) | ammonium hydrogenphosphate | as per test |
| 406 | (I-1-14) | ammonium dihydrogenphosphate | as per test |
| 407 | (I-1-14) | ammonium carbonate | as per test |
| 408 | (I-1-14) | ammonium benzoate | as per test |
| 409 | (I-1-14) | ammonium sulphite | as per test |
| 410 | (I-1-14) | ammonium benzoate | as per test |
| 411 | (I-1-14) | ammonium hydrogenoxalate | as per test |
| 412 | (I-1-14) | ammonium hydrogencitrate | as per test |
| 413 | (I-1-14) | tetramethylammonium sulphate | as per test |
| 414 | (I-1-14) | tetramethylammonium lactate | as per test |
| 415 | (I-1-14) | tetramethylammonium nitrate | as per test |
| 416 | (I-1-14) | tetramethylammonium thiosulphate | as per test |
| 417 | (I-1-14) | tetramethylammonium thiocyanate | as per test |
| 418 | (I-1-14) | tetramethylammonium citrate | as per test |
| 419 | (I-1-14) | tetramethylammonium oxalate | as per test |
| 420 | (I-1-14) | tetramethylammonium formate | as per test |
| 421 | (I-1-14) | tetramethylammonium hydrogenphosphate | as per test |
| 422 | (I-1-14) | tetramethylammonium dihydrogenphosphate | as per test |
| 423 | (I-1-14) | tetraethylammonium sulphate | as per test |
| 424 | (I-1-14) | tetraethylammonium lactate | as per test |
| 425 | (I-1-14) | tetraethylammonium nitrate | as per test |
| 426 | (I-1-14) | tetraethylammonium thiosulphate | as per test |
| 427 | (I-1-14) | tetraethylammonium thiocyanate | as per test |
| 428 | (I-1-14) | tetraethylammonium citrate | as per test |
| 429 | (I-1-14) | tetraethylammonium oxalate | as per test |
| 430 | (I-1-14) | tetraethylammonium formate | as per test |
| 431 | (I-1-14) | tetraethylammonium hydrogenphosphate | as per test |
| 432 | (I-1-14) | tetraethylammonium dihydrogenphosphate | as per test |
| 433 | (I-1-18) | ammonium sulphate | as per test |
| 434 | (I-1-18) | ammonium lactate | as per test |
| 435 | (I-1-18) | ammonium nitrate | as per test |
| 436 | (I-1-18) | ammonium thiosulphate | as per test |
| 437 | (I-1-18) | ammonium thiocyanate | as per test |
| 438 | (I-1-18) | ammonium citrate | as per test |
| 439 | (I-1-18) | ammonium oxalate | as per test |
| 440 | (I-1-18) | ammonium formate | as per test |
| 441 | (I-1-18) | ammonium hydrogenphosphate | as per test |
| 442 | (I-1-18) | ammonium dihydrogenphosphate | as per test |
| 443 | (I-1-18) | ammonium carbonate | as per test |
| 444 | (I-1-18) | ammonium benzoate | as per test |
| 445 | (I-1-18) | ammonium sulphite | as per test |
| 446 | (I-1-18) | ammonium benzoate | as per test |
| 447 | (I-1-18) | ammonium hydrogenoxalate | as per test |
| 448 | (I-1-18) | ammonium hydrogencitrate | as per test |
| 449 | (I-1-18) | tetramethylammonium sulphate | as per test |
| 450 | (I-1-18) | tetramethylammonium lactate | as per test |
| 451 | (I-1-18) | tetramethylammonium nitrate | as per test |
| 452 | (I-1-18) | tetramethylammonium thiosulphate | as per test |
| 453 | (I-1-18) | tetramethylammonium thiocyanate | as per test |
| 454 | (I-1-18) | tetramethylammonium citrate | as per test |
| 455 | (I-1-18) | tetramethylammonium oxalate | as per test |
| 456 | (I-1-18) | tetramethylammonium formate | as per test |
| 457 | (I-1-18) | tetramethylammonium hydrogenphosphate | as per test |
| 458 | (I-1-18) | tetramethylammonium dihydrogenphosphate | as per test |
| 459 | (I-1-18) | tetraethylammonium sulphate | as per test |
| 460 | (I-1-18) | tetraethylammonium lactate | as per test |
| 461 | (I-1-18) | tetraethylammonium nitrate | as per test |
| 462 | (I-1-18) | tetraethylammonium thiosulphate | as per test |
| 463 | (I-1-18) | tetraethylammonium thiocyanate | as per test |
| 464 | (I-1-18) | tetraethylammonium citrate | as per test |
| 465 | (I-1-18) | tetraethylammonium oxalate | as per test |
| 466 | (I-1-18) | tetraethylammonium formate | as per test |
| 467 | (I-1-18) | tetraethylammonium hydrogenphosphate | as per test |
| 468 | (I-1-18) | tetraethylammonium dihydrogenphosphate | as per test |
| 469 | (I-1-19) | ammonium sulphate | as per test |
| 470 | (I-1-19) | ammonium lactate | as per test |
| 471 | (I-1-19) | ammonium nitrate | as per test |
| 472 | (I-1-19) | ammonium thiosulphate | as per test |
| 473 | (I-1-19) | ammonium thiocyanate | as per test |
| 474 | (I-1-19) | ammonium citrate | as per test |
| 475 | (I-1-19) | ammonium oxalate | as per test |
| 476 | (I-1-19) | ammonium formate | as per test |
| 477 | (I-1-19) | ammonium hydrogenphosphate | as per test |
| 478 | (I-1-19) | ammonium dihydrogenphosphate | as per test |
| 479 | (I-1-19) | ammonium carbonate | as per test |
| 480 | (I-1-19) | ammonium benzoate | as per test |
| 481 | (I-1-19) | ammonium sulphite | as per test |
| 482 | (I-1-19) | ammonium benzoate | as per test |
| 483 | (I-1-19) | ammonium hydrogenoxalate | as per test |
| 484 | (I-1-19) | ammonium hydrogencitrate | as per test |
| 485 | (I-1-19) | tetramethylammonium sulphate | as per test |
| 486 | (I-1-19) | tetramethylammonium lactate | as per test |
| 487 | (I-1-19) | tetramethylammonium nitrate | as per test |
| 488 | (I-1-19) | tetramethylammonium thiosulphate | as per test |
| 489 | (I-1-19) | tetramethylammonium thiocyanate | as per test |
| 490 | (I-1-19) | tetramethylammonium citrate | as per test |
| 491 | (I-1-19) | tetramethylammonium oxalate | as per test |
| 492 | (I-1-19) | tetramethylammonium formate | as per test |
| 493 | (I-1-19) | tetramethylammonium hydrogenphosphate | as per test |
| 494 | (I-1-19) | tetramethylammonium dihydrogenphosphate | as per test |
| 495 | (I-1-19) | tetraethylammonium sulphate | as per test |
| 496 | (I-1-19) | tetraethylammonium lactate | as per test |
| 497 | (I-1-19) | tetraethylammonium nitrate | as per test |
| 498 | (I-1-19) | tetraethylammonium thiosulphate | as per test |
| 499 | (I-1-19) | tetraethylammonium thiocyanate | as per test |
| 500 | (I-1-19) | tetraethylammonium citrate | as per test |
| 501 | (I-1-19) | tetraethylammonium oxalate | as per test |
| 502 | (I-1-19) | tetraethylammonium formate | as per test |
| 503 | (I-1-19) | tetraethylammonium hydrogenphosphate | as per test |
| 504 | (I-1-19) | tetraethylammonium dihydrogenphosphate | as per test |
| 505 | (I-1-20) | ammonium sulphate | as per test |
| 506 | (I-1-20) | ammonium lactate | as per test |
| 507 | (I-1-20) | ammonium nitrate | as per test |
| 508 | (I-1-20) | ammonium thiosulphate | as per test |
| 509 | (I-1-20) | ammonium thiocyanate | as per test |
| 510 | (I-1-20) | ammonium citrate | as per test |
| 511 | (I-1-20) | ammonium oxalate | as per test |
| 512 | (I-1-20) | ammonium formate | as per test |
| 513 | (I-1-20) | ammonium hydrogenphosphate | as per test |
| 514 | (I-1-20) | ammonium dihydrogenphosphate | as per test |
| 515 | (I-1-20) | ammonium carbonate | as per test |
| 516 | (I-1-20) | ammonium benzoate | as per test |
| 517 | (I-1-20) | ammonium sulphite | as per test |
| 518 | (I-1-20) | ammonium benzoate | as per test |
| 519 | (I-1-20) | ammonium hydrogenoxalate | as per test |
| 520 | (I-1-20) | ammonium hydrogencitrate | as per test |
| 521 | (I-1-20) | tetramethylammonium sulphate | as per test |
| 522 | (I-1-20) | tetramethylammonium lactate | as per test |
| 523 | (I-1-20) | tetramethylammonium nitrate | as per test |
| 524 | (I-1-20) | tetramethylammonium thiosulphate | as per test |
| 525 | (I-1-20) | tetramethylammonium thiocyanate | as per test |
| 526 | (I-1-20) | tetramethylammonium citrate | as per test |
| 527 | (I-1-20) | tetramethylammonium oxalate | as per test |
| 528 | (I-1-20) | tetramethylammonium formate | as per test |
| 529 | (I-1-20) | tetramethylammonium hydrogenphosphate | as per test |
| 530 | (I-1-20) | tetramethylammonium dihydrogenphosphate | as per test |
| 531 | (I-1-20) | tetraethylammonium sulphate | as per test |
| 532 | (I-1-20) | tetraethylammonium lactate | as per test |
| 533 | (I-1-20) | tetraethylammonium nitrate | as per test |
| 534 | (I-1-20) | tetraethylammonium thiosulphate | as per test |
| 535 | (I-1-20) | tetraethylammonium thiocyanate | as per test |
| 536 | (I-1-20) | tetraethylammonium citrate | as per test |
| 537 | (I-1-20) | tetraethylammonium oxalate | as per test |
| 538 | (I-1-20) | tetraethylammonium formate | as per test |
| 539 | (I-1-20) | tetraethylammonium hydrogenphosphate | as per test |
| 540 | (I-1-20) | tetraethylammonium dihydrogenphosphate | as per test |
| 541 | (I-1-21) | ammonium sulphate | as per test |
| 542 | (I-1-21) | ammonium lactate | as per test |
| 543 | (I-1-21) | ammonium nitrate | as per test |
| 544 | (I-1-21) | ammonium thiosulphate | as per test |
| 545 | (I-1-21) | ammonium thiocyanate | as per test |
| 546 | (I-1-21) | ammonium citrate | as per test |
| 547 | (I-1-21) | ammonium oxalate | as per test |
| 548 | (I-1-21) | ammonium formate | as per test |
| 549 | (I-1-21) | ammonium hydrogenphosphate | as per test |
| 550 | (I-1-21) | ammonium dihydrogenphosphate | as per test |
| 551 | (I-1-21) | ammonium carbonate | as per test |
| 552 | (I-1-21) | ammonium benzoate | as per test |
| 553 | (I-1-21) | ammonium sulphite | as per test |
| 554 | (I-1-21) | ammonium benzoate | as per test |
| 555 | (I-1-21) | ammonium hydrogenoxalate | as per test |
| 556 | (I-1-21) | ammonium hydrogencitrate | as per test |
| 557 | (I-1-21) | tetramethylammonium sulphate | as per test |
| 558 | (I-1-21) | tetramethylammonium lactate | as per test |
| 559 | (I-1-21) | tetramethylammonium nitrate | as per test |
| 560 | (I-1-21) | tetramethylammonium thiosulphate | as per test |
| 561 | (I-1-21) | tetramethylammonium thiocyanate | as per test |
| 562 | (I-1-21) | tetramethylammonium citrate | as per test |
| 563 | (I-1-21) | tetramethylammonium oxalate | as per test |
| 564 | (I-1-21) | tetramethylammonium formate | as per test |
| 565 | (I-1-21) | tetramethylammonium hydrogenphosphate | as per test |
| 566 | (I-1-21) | tetramethylammonium dihydrogenphosphate | as per test |
| 567 | (I-1-21) | tetraethylammonium sulphate | as per test |
| 568 | (I-1-21) | tetraethylammonium lactate | as per test |
| 569 | (I-1-21) | tetraethylammonium nitrate | as per test |
| 570 | (I-1-21) | tetraethylammonium thiosulphate | as per test |
| 571 | (I-1-21) | tetraethylammonium thiocyanate | as per test |
| 572 | (I-1-21) | tetraethylammonium citrate | as per test |
| 573 | (I-1-21) | tetraethylammonium oxalate | as per test |
| 574 | (I-1-21) | tetraethylammonium formate | as per test |
| 575 | (I-1-21) | tetraethylammonium hydrogenphosphate | as per test |
| 576 | (I-1-21) | tetraethylammonium dihydrogenphosphate | as per test |
| 577 | (I-1-22) | ammonium sulphate | as per test |
| 578 | (I-1-22) | ammonium lactate | as per test |
| 579 | (I-1-22) | ammonium nitrate | as per test |
| 580 | (I-1-22) | ammonium thiosulphate | as per test |
| 581 | (I-1-22) | ammonium thiocyanate | as per test |
| 582 | (I-1-22) | ammonium citrate | as per test |
| 583 | (I-1-22) | ammonium oxalate | as per test |
| 584 | (I-1-22) | ammonium formate | as per test |
| 585 | (I-1-22) | ammonium hydrogenphosphate | as per test |
| 586 | (I-1-22) | ammonium dihydrogenphosphate | as per test |
| 587 | (I-1-22) | ammonium carbonate | as per test |
| 588 | (I-1-22) | ammonium benzoate | as per test |
| 589 | (I-1-22) | ammonium sulphite | as per test |
| 590 | (I-1-22) | ammonium benzoate | as per test |
| 591 | (I-1-22) | ammonium hydrogenoxalate | as per test |
| 592 | (I-1-22) | ammonium hydrogencitrate | as per test |
| 593 | (I-1-22) | tetramethylammonium sulphate | as per test |
| 594 | (I-1-22) | tetramethylammonium lactate | as per test |
| 595 | (I-1-22) | tetramethylammonium nitrate | as per test |
| 596 | (I-1-22) | tetramethylammonium thiosulphate | as per test |
| 597 | (I-1-22) | tetramethylammonium thiocyanate | as per test |
| 598 | (I-1-22) | tetramethylammonium citrate | as per test |
| 599 | (I-1-22) | tetramethylammonium oxalate | as per test |
| 600 | (I-1-22) | tetramethylammonium formate | as per test |
| 601 | (I-1-22) | tetramethylammonium hydrogenphosphate | as per test |
| 602 | (I-1-22) | tetramethylammonium dihydrogenphosphate | as per test |
| 603 | (I-1-22) | tetraethylammonium sulphate | as per test |
| 604 | (I-1-22) | tetraethylammonium lactate | as per test |
| 605 | (I-1-22) | tetraethylammonium nitrate | as per test |
| 606 | (I-1-22) | tetraethylammonium thiosulphate | as per test |
| 607 | (I-1-22) | tetraethylammonium thiocyanate | as per test |
| 608 | (I-1-22) | tetraethylammonium citrate | as per test |
| 609 | (I-1-22) | tetraethylammonium oxalate | as per test |
| 610 | (I-1-22) | tetraethylammonium formate | as per test |
| 611 | (I-1-22) | tetraethylammonium hydrogenphosphate | as per test |
| 612 | (I-1-22) | tetraethylammonium dihydrogenphosphate | as per test |
| 613 | (I-1-23) | ammonium sulphate | as per test |
| 614 | (I-1-23) | ammonium lactate | as per test |
| 615 | (I-1-23) | ammonium nitrate | as per test |
| 616 | (I-1-23) | ammonium thiosulphate | as per test |
| 617 | (I-1-23) | ammonium thiocyanate | as per test |
| 618 | (I-1-23) | ammonium citrate | as per test |
| 619 | (I-1-23) | ammonium oxalate | as per test |
| 620 | (I-1-23) | ammonium formate | as per test |
| 621 | (I-1-23) | ammonium hydrogenphosphate | as per test |
| 622 | (I-1-23) | ammonium dihydrogenphosphate | as per test |
| 623 | (I-1-23) | ammonium carbonate | as per test |
| 624 | (I-1-23) | ammonium benzoate | as per test |
| 625 | (I-1-23) | ammonium sulphite | as per test |
| 626 | (I-1-23) | ammonium benzoate | as per test |
| 627 | (I-1-23) | ammonium hydrogenoxalate | as per test |
| 628 | (I-1-23) | ammonium hydrogencitrate | as per test |
| 629 | (I-1-23) | tetramethylammonium sulphate | as per test |
| 630 | (I-1-23) | tetramethylammonium lactate | as per test |
| 631 | (I-1-23) | tetramethylammonium nitrate | as per test |
| 632 | (I-1-23) | tetramethylammonium thiosulphate | as per test |
| 633 | (I-1-23) | tetramethylammonium thiocyanate | as per test |
| 634 | (I-1-23) | tetramethylammonium citrate | as per test |
| 635 | (I-1-23) | tetramethylammonium oxalate | as per test |
| 636 | (I-1-23) | tetramethylammonium formate | as per test |
| 637 | (I-1-23) | tetramethylammonium hydrogenphosphate | as per test |
| 638 | (I-1-23) | tetramethylammonium dihydrogenphosphate | as per test |
| 639 | (I-1-23) | tetraethylammonium sulphate | as per test |
| 640 | (I-1-23) | tetraethylammonium lactate | as per test |
| 641 | (I-1-23) | tetraethylammonium nitrate | as per test |
| 642 | (I-1-23) | tetraethylammonium thiosulphate | as per test |
| 643 | (I-1-23) | tetraethylammonium thiocyanate | as per test |
| 644 | (I-1-23) | tetraethylammonium citrate | as per test |
| 645 | (I-1-23) | tetraethylammonium oxalate | as per test |
| 646 | (I-1-23) | tetraethylammonium formate | as per test |
| 647 | (I-1-23) | tetraethylammonium hydrogenphosphate | as per test |
| 648 | (I-1-23) | tetraethylammonium dihydrogenphosphate | as per test |
| 649 | (I-1-24) | ammonium sulphate | as per test |
| 650 | (I-1-24) | ammonium lactate | as per test |
| 651 | (I-1-24) | ammonium nitrate | as per test |
| 652 | (I-1-24) | ammonium thiosulphate | as per test |
| 653 | (I-1-24) | ammonium thiocyanate | as per test |
| 654 | (I-1-24) | ammonium citrate | as per test |
| 655 | (I-1-24) | ammonium oxalate | as per test |
| 656 | (I-1-24) | ammonium formate | as per test |
| 657 | (I-1-24) | ammonium hydrogenphosphate | as per test |
| 658 | (I-1-24) | ammonium dihydrogenphosphate | as per test |
| 659 | (I-1-24) | ammonium carbonate | as per test |
| 660 | (I-1-24) | ammonium benzoate | as per test |
| 661 | (I-1-24) | ammonium sulphite | as per test |
| 662 | (I-1-24) | ammonium benzoate | as per test |
| 663 | (I-1-24) | ammonium hydrogenoxalate | as per test |
| 664 | (I-1-24) | ammonium hydrogencitrate | as per test |
| 665 | (I-1-24) | tetramethylammonium sulphate | as per test |
| 666 | (I-1-24) | tetramethylammonium lactate | as per test |
| 667 | (I-1-24) | tetramethylammonium nitrate | as per test |
| 668 | (I-1-24) | tetramethylammonium thiosulphate | as per test |
| 669 | (I-1-24) | tetramethylammonium thiocyanate | as per test |
| 670 | (I-1-24) | tetramethylammonium citrate | as per test |
| 671 | (I-1-24) | tetramethylammonium oxalate | as per test |
| 672 | (I-1-24) | tetramethylammonium formate | as per test |
| 673 | (I-1-24) | tetramethylammonium hydrogenphosphate | as per test |
| 674 | (I-1-24) | tetramethylammonium dihydrogenphosphate | as per test |
| 675 | (I-1-24) | tetraethylammonium sulphate | as per test |
| 676 | (I-1-24) | tetraethylammonium lactate | as per test |
| 677 | (I-1-24) | tetraethylammonium nitrate | as per test |
| 678 | (I-1-24) | tetraethylammonium thiosulphate | as per test |
| 679 | (I-1-24) | tetraethylammonium thiocyanate | as per test |
| 680 | (I-1-24) | tetraethylammonium citrate | as per test |
| 681 | (I-1-24) | tetraethylammonium oxalate | as per test |
| 682 | (I-1-24) | tetraethylammonium formate | as per test |
| 683 | (I-1-24) | tetraethylammonium hydrogenphosphate | as per test |
| 684 | (I-1-24) | tetraethylammonium dihydrogenphosphate | as per test |

Crop protection compositions according to the invention may also comprise further components, for example, surfactants and/or dispersing auxiliaries or emulsifiers.

Suitable nonionic surfactants and/or dispersing auxiliaries include all substances of this type that can usually be used in agrochemical compositions. Polyethylene oxide/polypropylene oxide block copolymers, polyethylene glycol ethers of straight-chain alcohols, reaction products of fatty acids with ethylene oxide and/or propylene oxide, furthermore polyvinyl alcohol, polyvinyl pyrrolidone, copolymers of polyvinyl alcohol and polyvinyl pyrrolidone and copolymers of (meth)acrylic acid and (meth)acrylic esters, and additionally alkyl ethoxylates and alkylaryl ethoxylates, which optionally may be phosphated and optionally may be neutralized with bases, examples of which that may be mentioned being sorbitol ethoxylates, and also polyoxyalkyleneamine derivatives may be mentioned as being preferred.

Suitable anionic surfactants are all substances of this type that can usually be used in agrochemical compositions. Preference is given to alkali metal salts and alkaline earth metal salts of alkylsulphonic acids or alkylarylsulphonic acids.

A further preferred group of anionic surfactants and/or dispersing auxiliaries are the following salts that are of low solubility in plant oil: salts of polystyrenesulphonic acids, salts of polyvinylsulphonic acids, salts of naphthalenesulphonic acid/formaldehyde condensation products, salts of condensation products of naphthalenesulphonic acid, phenolsulphonic acid and formaldehyde, and salts of lignosulphonic acid.

Suitable additives which may be included in the formulations according to the invention are emulsifiers, foam inhibitors, preservatives, antioxidants, colorants and inert filling materials.

Preferred emulsifiers are ethoxylated nonylphenols, reaction products of alkylphenols with ethylene oxide and/or propylene oxide, ethoxylated arylalkylphenols, and also ethoxylated and propoxylated arylalkylphenols, and also sulphated or phosphated arylalkyl ethoxylates and/or arylalkyl ethoxypropoxylates, mention being made by way of example of sorbitan derivatives, such as polyethylene oxide sorbitan fatty acid esters and sorbitan fatty acid esters.

### EXAMPLES

### Myzus persicae Test (MYZUPE)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. If addition of ammonium salts or ammonium salts and penetrant is required, the appropriate amount is added by pipette after dilution of the respective finished preparation solution.

Bell pepper plants (Capsicum annuum) which are heavily infested by the Green peach aphid (Myzus persicaei) are treated by spraying with the active compound preparation of the desired concentration.

After the desired period of time, the kill in % is determined. 100% means that all aphids have been killed; 0% means that none of the aphids have been killed.

In this test, for example, the following embodiments of the invention show good activity: see table below

MYZUPE

| Compound | ppm | % 6 d |
|---|---|---|
| (I-1-20) + AS | — | — |
| according to the invention | | |
| (I-1-20) + AS + penetrant | 0.8 | 95 |
| according to the invention | | |
| (I-1-20) | 0.8 | 0 |
| prior art | | |
| (I-1-3) + AS + penetrant | 0.8 | 70 |
| according to the invention | | |
| (I-1-3) | 0.8 | 0 |
| prior art | | |

Aphis gossypii Test (APHIGO)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. If addition of ammonium salts or ammonium salts and penetrant is required, the appropriate amount is added by pipette after dilution of the respective finished preparation solution.

Cotton leaves (Gossypium hirsutum) which are heavily infested by the cotton aphid (Aphis gossypii) are sprayed with an active compound preparation of the desired concentration.

After the desired period of time, the kill in % is determined. 100% means that all aphids have been killed; 0% means that none of the aphids have been killed.

In this test, for example, the following embodiments of the invention show good activity: see table below

APHIGO

| Compound | ppm | % 6 d |
|---|---|---|
| (I-1-20) + AS | 0.8 | 95 |
| according to the invention | | |
| (I-1-20) + AS + penetrant | 0.8 | 95 |
| according to the invention | | |
| (I-1-20) | 0.8 | 0 |
| prior art | | |
| (I-1-3) + AS + penetrant | 0.8 | 80 |
| according to the invention | | |
| (I-1-3) | 0.8 | 0 |
| prior art | | |

1. Composition comprising
at least one insecticidally or acaricidally active compound from the class of the anthranilamides
at least one salt of formula (II) 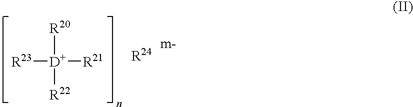
in which
D represents nitrogen or phosphorus,
R²⁰, R²¹, R²² and R²³ independently of one another represent hydrogen or in each case optionally substituted C₁-C₈-alkyl or mono- or polyunsaturated, optionally substituted C₁-C₈-alkylene, where the substituents may be selected from the group consisting of halogen, nitro and cyano,
m represents 1, 2, 3 or 4,
R²⁴ represents an inorganic or organic anion.

2. Composition according to claim 1, wherein the active compound is a compound of the formula (I) 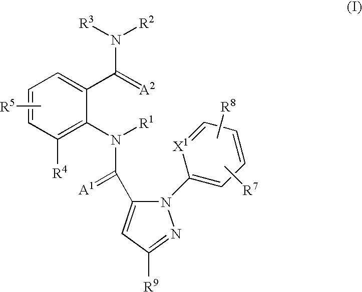
in which
A¹ and A² independently of one another represent oxygen or sulphur,
X¹ represents N or CR¹⁰,
R¹ represents hydrogen or represents in each case optionally mono- or polysubstituted C₁-C₆-alkyl, C₂-C₆-alkenyl, C₂-C₆-alkynyl or C₃-C₆-cycloalkyl, where the substituents independently of one another may be selected from the group consisting of R⁶, halogen, cyano, nitro, hydroxyl, C₁-C₄-alkoxy, C₁-C₄-alkylthio, C₁-C₄-alkylsulphinyl, C₁-C₄-alkylsulphonyl, C₂-C₄-alkoxycarbonyl, C₁-C₄-alkylamino, C₂-C₈-dialkylamino, C₃-C₆-cycloalkylamino, (C₁-C₄-alkyl)C₃-C₆-cycloalkylamino and R¹¹,
R² represents hydrogen, C₁-C₆-alkyl, C₂-C₆-alkenyl, C₂-C₆-alkynyl, C₃-C₆-cycloalkyl, C₁-C₄-alkoxy, C₁-C₄-alkylamino, C₂-C₈-dialkylamino, C₃-C₆-cycloalkylamino, C₂-C₆-alkoxycarbonyl or C₂-C₆-alkylcarbonyl,
R³ represents hydrogen, R¹¹ or represents in each case optionally mono- or polysubstituted C₁-C₆-alkyl, C₂-C₆-alkenyl, C₂-C₆-alkynyl, C₃-C₆-cycloalkyl, where the substituents independently of one another may be selected from the group consisting of R⁶, halogen, cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₂-C₆-alkoxycarbonyl, C₂-C₆-alkylcarbonyl, C₃-C₆-trialkylsilyl, R¹¹, phenyl, phenoxy or a 5- or 6-membered heteroaromatic ring, where each phenyl, phenoxy and 5- or 6-membered heteroaromatic ring may optionally be substituted and where the substituents independently of one another may be selected from the group consisting of one to three radicals W and one or more radicals R¹², or
R² and R³ may be attached to one another and form the ring M,
R⁴ represents hydrogen, C₁-C₆-alkyl, C₂-C₆-alkenyl, C₂-C₆-alkynyl, C₃-C₆-cycloalkyl, C₁-C₆-haloalkyl, C₂-C₆-haloalkenyl, C₂-C₆-haloalkynyl, C₃-C₆-halocycloalkyl, halogen, cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, C₃-C₆-cycloalkylamino, C₃-C₆-trialkylsilyl or represents in each case optionally mono- or polysubstituted phenyl, benzyl or phenoxy, where the substituents independently of one another may be selected from the group consisting of C₁-C₄-alkyl, C₂-C₄-alkenyl, C₂-C₄-alkynyl, C₃-C₆-cyclalkyl, C₁-C₄-haloalkyl, C₂-C₄-haloalkenyl, C₂-C₄-haloalkynyl, C₃-C₆-halocycloalkyl, halogen, cyano, nitro, C₁-C₄-alkoxy, C₁-C₄-halo-alkoxy, C₁-C₄-alkylthio, C₁-C₄-alkylsulphinyl, C₁-C₄-alkylsulphonyl, C₁-C₄-alkylamino, C₂-C₈-dialkylamino, C₃-C₆-cycloalkylamino, C₃-C₆-(alkyl)cycloalkylamino, C₂-C₄-alkylcarbonyl, C₂-C₆-alkoxycarbonyl, C₂-C₆-alkylaminocarbonyl, C₃-C₈-dialkylaminocarbonyl and C₃-C₆-trialkylsilyl,
R⁵ and R⁸ each independently of one another represent hydrogen, cyano, halogen or represent in each case optionally substituted C₁-C₄-alkyl, C₁-C₄-haloalkyl, R¹², G, J, —OJ, —OG, —S(O)ₚ-J, —S(O)ₚ-G, —S(O)ₚ-phenyl, where the substituents independently of one another may be selected from the group consisting of one to three radicals W or of R¹², C₁-C₁₀-alkyl, C₂-C₆-alkenyl, C₂-C₆-alkynyl, C₁-C₄-alkoxy or C₁-C₄-alkylhio, where each substituent may be substituted by one or more substituents independently of one another selected from the group consisting of G, J, R⁶, halogen, cyano, nitro, amino, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, C₃-C₆-trialkylsilyl, phenyl and phenoxy, where each phenyl or phenoxy ring may optionally be substituted and where the substituents independently of one another may be selected from the group consisting of one to three radicals W and one or more radicals R¹²,
G in each case independently represents a 5- or 6-membered non-aromatic carbocyclic or heterocyclic ring which may optionally contain one or two ring members from the group consisting of C(═O), SO and S(═O)₂ and which may optionally be substituted by one to four substituents independently of one another selected from the group consisting of C₁-C₂-alkyl, halogen, cyano, nitro and C₁-C₂-alkoxy, or independently represents C₂-C₆-alkenyl, C₂-C₆-alkynyl, C₃-C₇-cycloalkyl, (cyano)C₃-C₇-cycloalkyl, (C₁-C₄-alkyl)C₃-C₆-cycloalkyl, (C₃-C₆-cycloalkyl)C₁-C₄-alkyl, where each cycloalkyl, (alkyl)cycloalkyl and (cycloalkyl)alkyl may optionally be substituted by one or more halogen atoms,
J in each case independently represents an optionally substituted 5- or 6-membered heteroaromatic ring, where the substituents independently of one another may be selected from the group consisting of one to three radicals W and one or more radicals R¹²,
R⁶ independently represents —C(=E¹)R¹⁹, -LC(=E¹)R¹⁹, —C(=E¹)LR¹⁹, -LC(=E¹)LR¹⁹, —OP(═O)(OR¹⁹)₂, —SO₂LR¹⁸ or -LSO₂LR¹⁹, where each E¹ independently represents O, S, N—R¹⁵, N—OR¹⁵, N—N(R¹⁵)₂, N—S═O, N—CN or N—NO₂,
R⁷ represents hydrogen, C₁-C₄-alkyl, C₁-C₄-haloalkyl, halogen, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl,
R⁹ represents C₁-C₄-haloalkyl, C₁-C₄-haloalkoxy, C₁-C₄-haloalkylsulphinyl or halogen,
R¹⁰ represents hydrogen, C₁-C₄-alkyl, C₁-C₄-haloalkyl, halogen, cyano or C₁-C₄-haloalkoxy,
R¹¹ in each case independently represents in each case optionally mono- to trisubstituted C₁-C₆-alkylthio, C₁-C₆-alkylsulphenyl, C₁-C₆-haloalkylhio, C₁-C₆-haloalkylsulphenyl, phenylthio or phenylsulphenyl, where the substituents independently of one another may be selected from the group consisting of W, —S(O)ₙN(R¹⁶)₂, —C(═O)R³, -L(C═O)R¹⁴, —S(C═O)LR⁴, —C(═O)LR¹³, —S(O)ₙNR¹³C(═O)R¹³, —S(O)ₙNR¹³C(═O)LR¹⁴ and —S(O)ₙNR¹³S(O)₂LR¹⁴,
L in each case independently represents O, NR¹⁸ or S,
R¹² in each case independently represents —B(OR¹⁷)₂, amino, SH, thiocyanato, C₃-C₈-trialkylsilyloxy, C₁-C₄-alkyl disulphide, —SF₅, —C(=E¹)R¹⁹, -LC(=E¹)R¹⁹, —C(=E¹)LR¹⁹, -LC(=E¹)LR¹⁹, —OP(═O)(OR¹⁹)₂, —SO₂LR¹⁹ or -LSO₂LR¹⁹,
Q represents O or S,
R¹³ in each case independently represents hydrogen or represents in each case optionally mono- or polysubstituted C₁-C₆-alkyl, C₂-C₆-alkenyl, C₂-C₆-alkynyl or C₃-C₆-cycloalkyl, where the substituents independently of one another may be selected from the group consisting of R⁶, halogen, cyano, nitro, hydroxyl, C₁-C₄-alkoxy, C₁-C₄-alkylsulphinyl, C₁-C₄-alkylsulphonyl, C₁-C₄-alkylamino, C₂-C₈-dialkylamino, C₃-C₆-cycloalkylamino and (C₁-C₄-alkyl)C₃-C₆-cycloalkylamino,
R¹⁴ in each case independently represents in each case optionally mono- or polysubstituted C₁-C₂₀-alkyl, C₂-C₂₀-alkenyl, C₂-C₂₀-alkynyl or C₃-C₆-cycloalkyl, where the substituents independently of one another may be selected from the group consisting of R⁶, halogen, cyano, nitro, hydroxyl, C₁-C₄-alkoxy, C₁-C₄-alkylsulphinyl, C₁-C₄-alkylsulphonyl, C₁-C₄-alkylamino, C₂-C₈-dialkylamino, C₃-C₆-cycloalkylamino and (C₁-C₄-alkyl)C₃-C₆-cycloalkylamino or represents optionally substituted phenyl, where the substituents independently of one another may be selected from the group consisting of one to three radicals W and one or more radicals R¹²
R¹⁵ in each case independently represents hydrogen or represents in each case optionally mono- or polysubstituted C₁-C₆-haloalkyl or C₁-C₆-alkyl, where the substituents independently of one another may be selected from the group consisting of cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, C₂-C₆-alkoxycarbonyl, C₂-C₆-alkylcarbonyl, C₃-C₆-trialkylsilyl and optionally substituted phenyl, where the substituents independently of one another may be selected from the group consisting of one to three radicals W and one or more radicals R¹², or N(R¹⁵)₂ represents a cycle which forms the ring M,
R¹⁶ represents C₁-C₁₂-alkyl or C₁-C₁₂-haloalkyl, or N(R¹⁶)₂ represents a cycle which forms the ring M,
R¹⁷ in each case independently represents hydrogen or C₁-C₄-alkyl, or B(OR¹⁷)₂ represents a ring in which the two oxygen atoms are linked via a chain of two or three carbon atoms which are optionally substituted by one or two substituents independently of one another selected from the group consisting of methyl and C₂-C₆-alkoxycarbonyl,
R¹⁸ in each case independently represents hydrogen, C₁-C₆-alkyl or C₁-C₆-haloalkyl, or N(R¹³)(R¹⁸) represents a cycle which forms the ring M,
R¹⁹ in each case independently represents hydrogen or represents in each case optionally mono- or polysubstituted C₁-C₆-alkyl, where the substituents independently of one another may be selected from the group consisting of cyano, nitro, hydroxyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylamino, C₂-C₈-dialkylamino, CO₂H, C₂-C₆-alkoxycarbonyl, C₂-C₆-alkylcarbonyl, C₃-C₆-trialkylsilyl and optionally substituted phenyl, where the substituents independently of one another may be selected from the group consisting of one to three radicals W, C₁-C₆-haloalkyl, C₃-C₆-cycloalkyl and phenyl or pyridyl, each of which is optionally mono- to trisubstituted by W,
M represents in each case an optionally mono- to tetrasubstituted ring which, in addition to the nitrogen atom attached to the substituent pair R¹³ and R¹⁸, (R¹⁵)₂ or (R¹⁶)₂ contains two to six carbon atoms and optionally additionally a further nitrogen, sulphur or oxygen atom and where the substituents independently of one another may be selected from the group consisting of C₁-C₂-alkyl, halogen, cyano, nitro and C₁-C₂-alkoxy,
W in each case independently represents C₁-C₄-alkyl, C₂-C₄-alkenyl, C₂-C₄-alkynyl, C₃-C₆-cycloalkyl, C₁-C₄-haloalkyl, C₂-C₄-haloalkenyl, C₂-C₄-haloalkynyl, C₃-C₆-halocycloalkyl, halogen, cyano, nitro, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylamino, C₂-C₈-dialkylamino, C₃-C₆-cycloalkylamino, (C₁-C₄-alkyl)C₃-C₆-cycloalkylamino, C₂-C₄-alkylcarbonyl, C₂-C₆-alkoxycarbonyl, CO₂H, C₂-C₆-alkylaminocarbonyl, C₃-C₈-dialkylaminocarbonyl or C₃-C₆-trialkylsilyl,
n in each case independently represents 0 or 1,
p in each case independently represents 0, 1 or 2,
and/or a salt of formula (I) and/or an N-oxide of a compound of the formula (I).

3. Composition according to claim 1, wherein the active compound is present in an amount from 0.5 to 50% by weight.

4. Composition according to claim 1, wherein D represents nitrogen.

5. Composition according to claim 4, wherein R²⁴ represents bicarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate, citrate or oxalate.

6. Composition according to claim 4, wherein R²⁴ represents carbonate, pentaborate, sulphite, benzoate, hydrogenoxalate, hydrogencitrate, methylsulphate or tetrafluoroborate.

7. Composition according to one or more of claim 1, further comprising at least one penetrant.

8. Composition according to claim 7, wherein the penetrant is i) a fatty alcohol alkoxylate of the formula (III)
R—O-(-AO)ᵥ—R′ (III)
in which
R represents straight-chain or branched alkyl having 4 to 20 carbon atoms,
R′ represents hydrogen, methyl, ethyl, n-propyl, isopropyl, n-butyl, isobutyl, t-butyl, n-pentyl or n-hexyl,
AO represents an ethylene oxide radical, a propylene oxide radical, a butylene oxide radical or represents mixtures of ethylene oxide and propylene oxide radicals or butylene oxide radicals and
v represents a numbers from 2 to 30,
and/or ii) a mineral or vegetable oil and/or iii) ester of a mineral or vegetable oil.

9. Composition according to claim 7, wherein the penetrant is an ester of a vegetable oil.

10. Composition according to claim 7, wherein the penetrant is rapeseed oil methyl ester.

11. Composition according to one or more of claim 7, wherein the penetrant is present in an amount from 1 to 95% by weight.

12. Method of controlling harmful insects, comprising applying a composition according to claim 1, diluted or undiluted to an insect or a habitat thereof in such an amount that an effective amount of the insecticidally active compounds contained therein is permitted to act on the insects or said habitat.

13. Method of increasing the activity of crop protection of an anthranilic acid diamide, comprising forming a ready-to-use spray liquor comprising a composition according to claim 1.

14. Method according to claim 12, wherein the spray liquor is prepared using a penetrant.

15. Method according to claim 13, wherein the salt of the formula (II) is present in a concentration of from 0.5 to 80 mmol/l.

16. Method according to claim 14, wherein the penetrant is present in a concentration of from 0.1 to 10 g/l.

17. Method according to claim 14, wherein the penetrant is present in a concentration of from 0.1 to 10 g/l and the salt of the formula (II) is present in a concentration of from 0.5 to 80 mmol/l.

18. A composition of claim 1, that is capable of increasing the activity of a crop protection comprising an anthranilic acid diamide compound wherein said composition comprises a ready-to-use crop protection spray liquor.

19. A composition according to claim 18, wherein the salt of the formula (II) is present in the ready-to-use crop protection spray liquor in a concentration of from 0.5 to 80 mmol/l.

20. A composition according to claim 18, further comprising a penetrant.
